(19) United States
(12) Patent Application Publication
(10) Pub. No.: US 2026/0047950 A1
(43) Pub. Date: Feb. 19, 2026

(54) APPARATUS AND METHOD FOR VARIABLE STIFFNESS ANKLE-FOOT ORTHOSIS

(71) Applicant: UNIVERSITY OF CENTRAL FLORIDA RESEARCH FOUNDATION, INC., Orlando, FL (US)

(72) Inventors: Hwan Choi, Orlando, FL (US); Sy Nguyen, Orlando, FL (US)

(21) Appl. No.: 19/294,768

(22) Filed: Aug. 8, 2025

Related U.S. Application Data
(60) Provisional application No. 63/684,004, filed on Aug. 16, 2024.

(51) Int. Cl. A61F 5/01

(52) U.S. Cl. CPC A61F 5/0127; A61F 2005/0155; A61F 2005/0167

(57) ABSTRACT

An apparatus is provided for variable stiffness in ankle-foot orthosis. The apparatus includes a first and second frame portion respectively attached to a shank and leg portion of an AFO frame. The second frame portion rotates relative to the first frame portion about a rotational axis. An elastic beam with segments is oriented in a radial direction. A motor is operatively connected to the segments to adjust an effective bend length of each segment about the rotational axis. A controller receives first data indicating a desired value of a level of stiffness of the apparatus and determines a desired value of the effective bend length of each segment based on the desired value of the level of stiffness. The controller transmits a signal to the motor to adjust the effective bend length to the desired value. A method employing the apparatus for variable stiffness in AFO is also provided.

### CROSS-REFERENCE TO RELATED APPLICATIONS

This application claims benefit of Provisional Application No. 63/684,004, filed Aug. 16, 2024, the entire contents of which is hereby incorporated by reference as if fully set forth herein, under 35 U.S.C. § 119 (e).

### BACKGROUND

A common medical device clinicians prescribe to improve walking mobility and gait in children with cerebral palsy (CP) and people with stroke is ankle foot orthosis (AFO). There are several different types of AFOs, each designed to address specific needs and conditions [1] [2] [3]. Generally, AFOs act parallel to the gastrocnemius, and adjusting its stiffness properly can reduce the gastrocnemius' excessive forces from the hyperexcitability [4]. Furthermore, different stiffness levels can impact the energy storage and return level as AFOs deform along with ankle joint dorsiflexion during walking. The proper return of energy can alleviate high metabolic costs by compensating for lack of propulsion and support during walking [5] [6] and ultimately improve walking in daily living [7]. At the same time, the AFOs can limit the ankle joint and reduce the muscle's eccentric contraction velocity to prevent the onset of hyperreflexia of the gastrocnemius and increase the gastrocnemius's operating length. AFOs can also assist in dorsiflexion by counteracting the downward movement of the foot and delivering an upward push. This is especially advantageous for those who have foot drop walking.

### SUMMARY

Ankle dorsiflexion stiffness is a device feature that provides support during the stance phase, balancing support and energy storage. Higher dorsiflexion stiffness restricts the device's energy storage capacity and the power generated during late stance push-off at the ankle. Conversely, lower stiffness offers less support but allows for more energy storage and push-off power [8]. AFOs are classified into two types. The simple one is passive AFOs, which are typically constructed of carbon fiber or thermal plastic (i.e., polypropylene) and have a high elasticity, and it allows them to store energy during mid-terminal stance due to the effect of body weight. Passive AFOs provide resistance to minimize muscle hyperreflexia and help compensate for the loss of gastrocnemius function. However, they are unable to alter ankle stiffness or offer energy return, limiting their ability to maintain and prescribe subject-specific ideal settings [9]. Adaptability to speed is the most important functional component of AFOs for people with cerebral palsy, with 94% of CP AFO users rating it crucial or extremely important. 87% of physicians saw adaptation to walking speed as crucial or very significant. [7]. Furthermore, doctors find it difficult to effectively balance the tradeoff when changing AFOs due to the effect of walking speed on stiffness requirements. Customizing stiffness for each user saves more energy than relying exclusively on manufacturer and medical recommendations. However, the optimal stiffness of an AFO is determined by the wearer's body mass, gait style, terrain, and speed fluctuations. [10] [11]. To overcome the disadvantages of the passive AFOs, on the other hand, several active AFO designs have been recently developed that can alter joint stiffness using a combination of elastic components and actuators to improve upon single-stiffness AFO. While motor-powered AFOs can be more practical compared to passive AFOs, they are still complex, noisy, bulky and heavy due to the size of the actuator and battery. Moreover, because batteries do not support long-range walking, children with CP may need to carry extra batteries. Hence, these limitations have prevented active AFOs from being used in daily life [12].

As a compromise between heavier active AFOs and standard passive AFOs, semi-active AFOs have been proposed [13] [14] [15] [16]. However, these AFOs use elastic bars or footplates with energy return that is insufficient to support walking due to energy loss from the elastomer [17] [18] [19]. Furthermore, their mechanisms often require greater energy consumption and have a slower rate of stiffness adjustments. Moreover, most AFOs recently used large actuators, thus requiring more energy and battery size. For example, Benjamin DeBoer et. Al. [20] developed an innovative, energy-efficient actuator for an active ankle-foot orthosis (AAFO). The AAFO operates on the principle of discrete non-linear stiffness. Multiple linear springs are compressed at specific displacement intervals to decrease the maximum mechanical power needed to operate the AAFO. The large actuators in this design are situated within a bounding box measuring 103×45×94 mm, and the total weight of this AAFO is 2.85 Kg. Also, increasing the number of springs and displacement offsets may result in a heavier actuator and larger system, as depicted in FIG. 1a. Sangjoon Jonathan Kim et. Al. [21] developed an active pneumatic remote transmission AFO. The system comprises an actuator coupled to an AFO, a costume compressor, a transmission system, and electronic components, as shown in FIG. 1b. The total weight of this design is 2.6 kg, making them bulkier and less appealing to wear. Also, an issue with this design is incorporating a specialized wearable compressor weighing 1.5 Kg, which the patient must carry as a backpack, restricting daily activities. Similarly, Junming Wang et. al. [22] designed an ankle-foot orthosis using a slack cable tendon mechanism and pneumatic actuator, as shown in FIG. 2a. The system's weight is approximately 1.6 kg. This method facilitates ankle dorsiflexion using a portable pump that weighs around 0.5 kg. The power system base is worn on the waist belt and houses the pump, valve, controller, and pressure sensor, making it large and bulky. Finally, Crey et. al. [23] recently developed a quasi-passive ankle-foot orthosis with variable stiffness using a lead screw and leaf spring, as shown in FIG. 2b. The design has a weight of 0.95 kg. The stiffness range of their designs for dorsiflexion and plantarflexion is 0.4-3.4 Nm/o and 0.2-0.8 Nm/o, respectively. However, the existing design has limited maximum dorsiflexion stiffness (greater than 4 Nm/o) that is required for rigid stiffness setting. [24].

Techniques are provided for a design to develop a simple, variable stiffness ankle-foot orthosis that can adjust the stiffness of the ankle joint during walking. The device can be easily attached to the conventional AFO metal frames or plastic shells. The variable stiffness module will be capable of adjusting the stiffness across a range of values (e.g., from about 1 Nm/° to about 4) Nm/°, but is light weight (e.g., about ˜1 kg) and has more energy efficiency to adjust the stiffness.

In a first set of embodiments, an apparatus is provided for providing variable stiffness for ankle-foot orthosis. The apparatus includes a first frame portion configured to attach to a shank portion of an ankle-foot orthosis (AFO) frame and a second frame portion configured to attach to a foot portion of the AFO frame. The second frame portion is configured to rotate relative to the first frame portion about a rotational axis when attached to the AFO frame. The apparatus also includes an elastic beam comprising a plurality of segments oriented with a first length in a radial direction that is orthogonal to the rotational axis. The apparatus also includes a motor operatively connected to the plurality of segments. The motor is configured to adjust an effective bend length of each of the plurality of segments about the rotational axis, where the effective bend length is less than or equal to the first length. The apparatus also includes a controller communicatively coupled with the motor and a memory including one or more sequences of instructions. The memory and the one or more sequences of instructions are configured to, with the controller, cause the apparatus to perform at least the following including to receive first data indicating a desired value of a level of stiffness of the apparatus about the rotational axis and to determine a desired value of the effective bend length of each of the plurality of segments based on the desired value of the level of stiffness. The memory and the sequences of instructions are also configured to, with the controller, cause the apparatus to transmit a signal to the motor to cause the motor to adjust the effective bend length to the desired value.

In a second set of embodiments, a method is provided for variable stiffness for ankle-foot orthosis. The method includes attaching a first frame portion to a shank portion of an ankle-foot orthosis (AFO) frame and attaching a second frame portion to a foot portion of the AFO frame. The second frame portion is configured to rotate relative to the first frame portion about a rotational axis when attached to the AFO frame. The method also includes providing an elastic beam comprising a plurality of segments oriented with a first length in a radial direction that is orthogonal to the rotational axis. The method also includes operatively connecting a motor to the plurality of segments, such that the motor is configured to adjust an effective bend length of each of the plurality of segments about the rotational axis. The effective bend length is less than or equal to the first length. The method also includes receiving, at a controller, first data indicating a desired value of a level of stiffness about the rotational axis. The method also includes determining, with the controller, a desired value of the effective bend length of each of the plurality of segments based on the desired value of the level of stiffness. The method also includes transmitting, from the controller, a signal the motor a signal to cause the motor to adjust the effective bend length to the desired value. The method also includes adjusting, with the motor, the effective bend length of each of the plurality of segments to the desired value.

In a third set of embodiments, a method is provided for optimizing a value of a parameter of the plurality of segments of the elastic beam in the apparatus in the first set of embodiments. The method includes a step of determining a desired range of stiffness for the apparatus in order to treat one or more subjects having one or more medical diagnoses. The method also includes a step of measuring a level of stiffness of one or more segments having one or more respective values of the parameter. The method also includes a step of selecting the plurality of segments for the apparatus and a respective value of the parameter for each of the plurality of segments of the apparatus, based on the measuring step, so that a range of the level of stiffness for the selected plurality of segments in the apparatus encompasses the desired range of stiffness.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is an image that illustrates an example of a perspective side view of a system for providing variable stiffness in an ankle-foot orthosis (AFO), according to an embodiment;

FIG. 1B is an image that illustrates an example of a perspective side view of a system for providing variable stiffness in an ankle-foot orthosis (AFO), according to an embodiment;

FIG. 1C is an image that illustrates an example of the system of FIGS. 1A and 1B attached to an ankle of a subject, according to an embodiment;

FIG. 1D is a diagram of a plurality of movement phases of a gait cycle of a subject, according to an embodiment;

FIG. 1E is an image that illustrate an example of a system used to calibrate the stress of the blade of the system of FIG. 1A, according to an embodiment;

FIG. 2A is an image that illustrates an example of an exploded view of the system of FIGS. 1A and 1B, according to an embodiment;

FIG. 2B is an image that illustrates an example of an exploded view of the apparatus of the system of FIGS. 1A and 1B, according to an embodiment;

FIG. 2C is an image that illustrates an example of a side view of the assembled system of FIGS. 1A and 1C, according to an embodiment;

FIG. 2D is an image that illustrates an example of an effective bend length of the elastic beam segments of FIGS. 2A and 2B based on the fulcrum position, according to an embodiment;

FIG. 2E are images that illustrate an example of a position of the cam gear, a position of the fulcrum and a stiffness of the AFO of the system of FIGS. 1A and 1B, according to an embodiment;

FIG. 2F are images that illustrate an example of a resistive moment imparted by the elastic beam segments of the apparatus in response to dorsiflexion and plantarflexion, according to an embodiment;

FIG. 2G is a block diagram that illustrates the components of the system of FIG. 2A, according to an embodiment;

FIGS. 2H through 2K are images that illustrate an example of a system for providing variable stiffness in an AFO, according to an embodiment;

FIG. 3A is a flow chart that illustrates an example method for providing variable stiffness to the apparatus of the system of FIGS. 1A and 1B, according to an embodiment;

FIG. 3B is a flow chart that illustrates an example method for optimizing a value of a parameter of a component of the apparatus of the system of FIGS. 1A and 1B, according to an embodiment;

FIG. 4 is a block diagram that illustrates a computer system upon which an embodiment of the invention may be implemented;

FIG. 5 illustrates a chip set upon which an embodiment of the invention may be implemented;

FIG. 6 illustrates a mobile terminal upon which an embodiment of the invention may be implemented;

FIGS. 7A and 7B are images that illustrate an example of a step motor and loadcell used to optimize one or more design parameters of the system of FIGS. 1A through 1C, according to an embodiment; and

FIGS. 8A through 8C are graphs that illustrate an example of stiffness data captured using the step motor and loadcell of FIGS. 7A and 7B for elastic beam segments of different diameter, according to an embodiment.

### DETAILED DESCRIPTION

A method and apparatus are described for providing variable stiffness in an ankle-foot orthosis (AFO). In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of ankle-foot orthosis (AFO). However, the invention is not limited to this context. In other embodiments the invention is described in a context of providing variable stiffness to any orthosis for any joint of the body, including ankle-foot but other joints such as knee or hip, for example.

### 1. System and Apparatus Overview

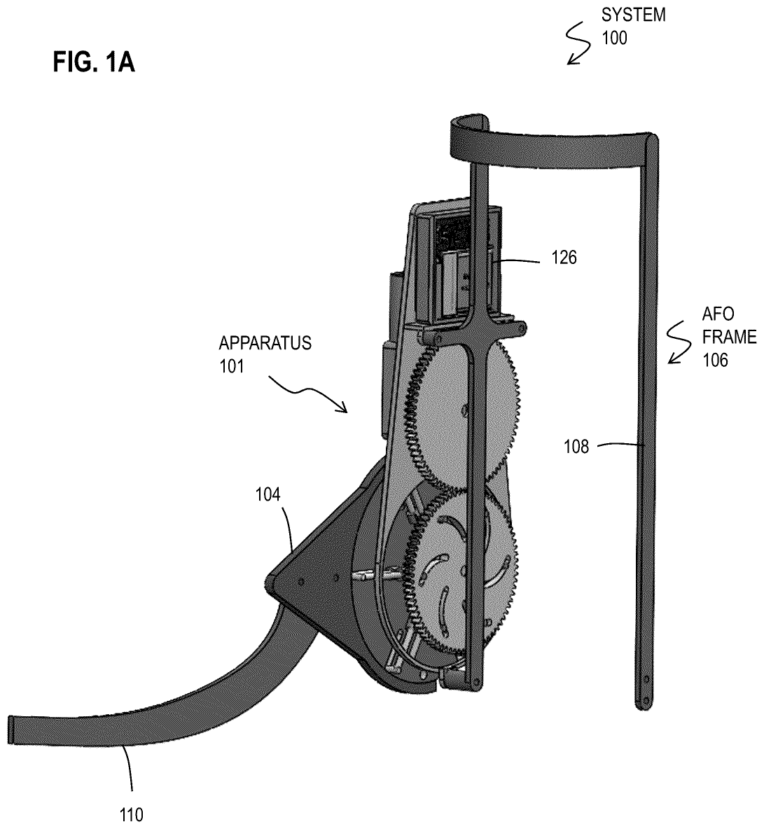

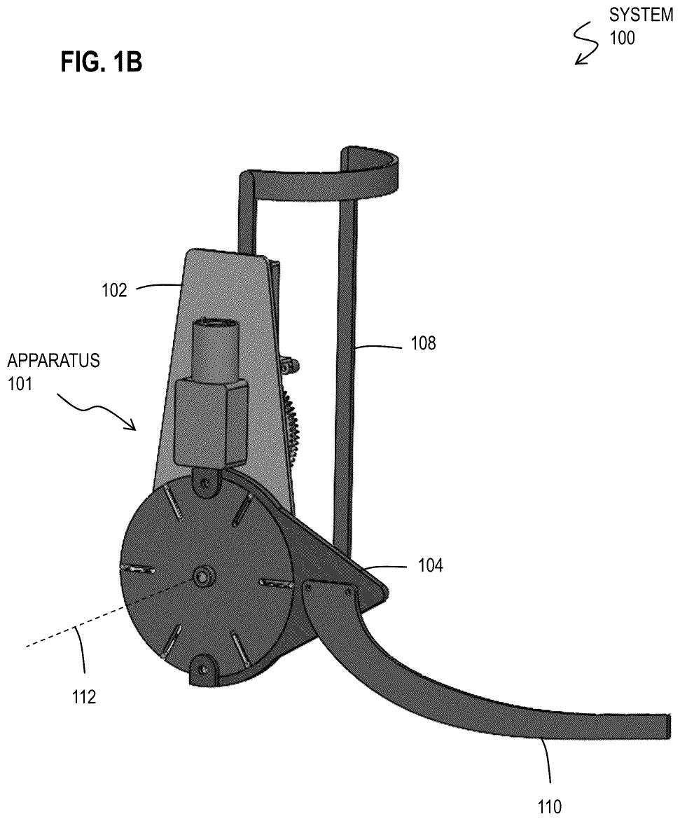

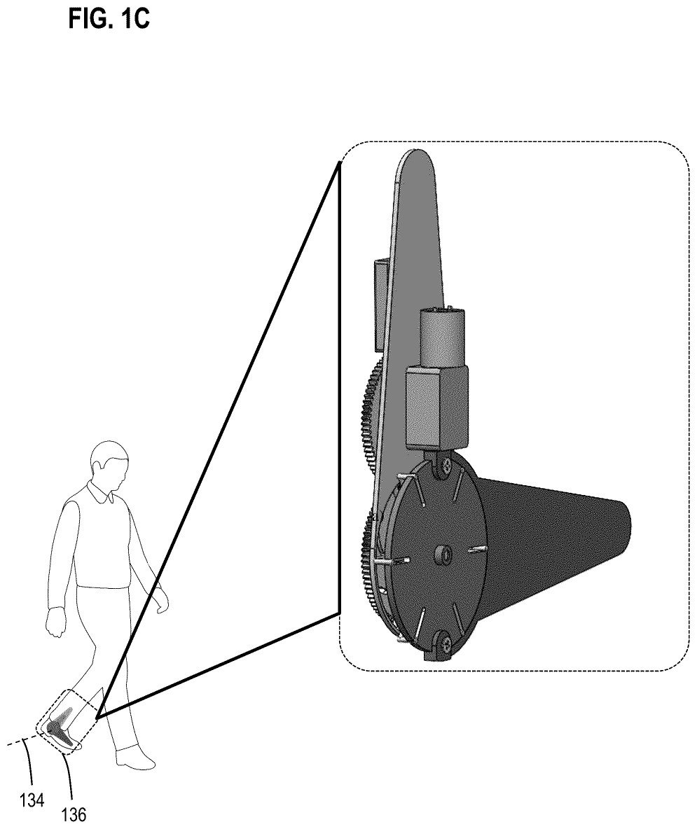

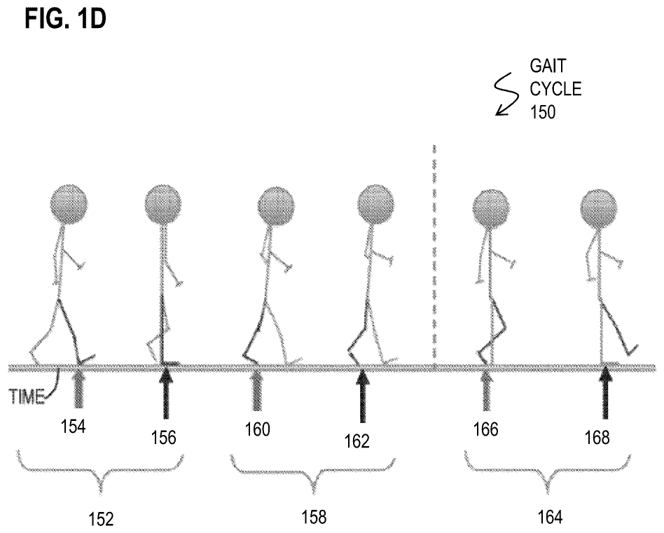

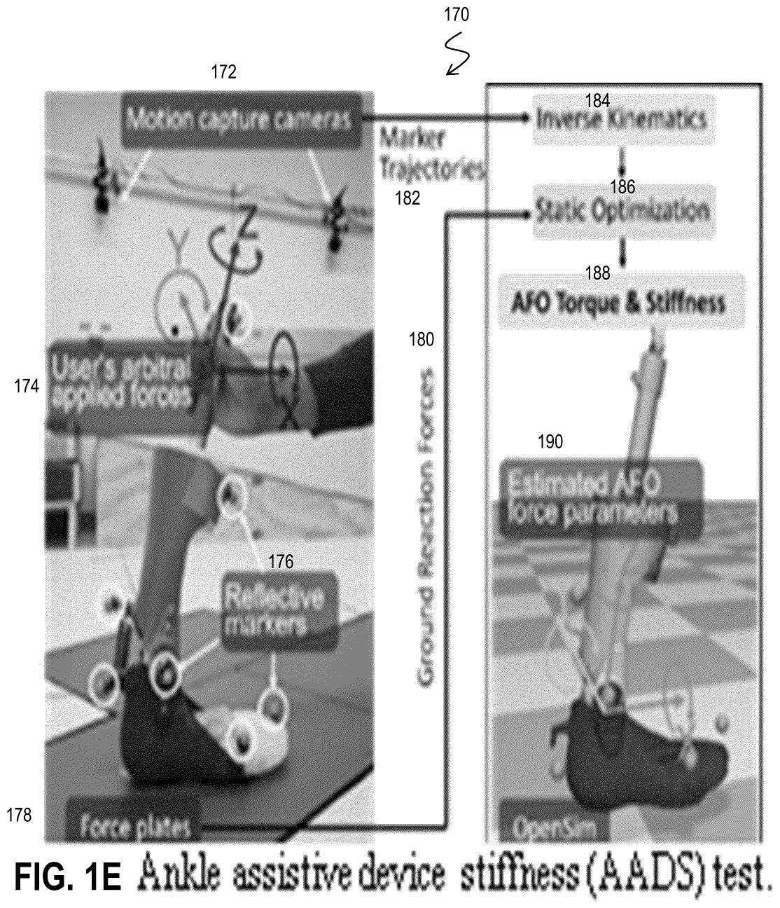

A discussion of an overall system that includes an apparatus and an ankle-foot orthosis (AFO) frame is now provided. FIG. 1A is an image that illustrates an example of a perspective side view of a system 100 for providing variable stiffness in an ankle-foot orthosis (AFO), according to an embodiment. FIG. 1B is an image that illustrates an example of a perspective side view of a system 100 for providing variable stiffness in an ankle-foot orthosis (AFO), according to an embodiment. As shown in FIGS. 1A and 1B, the system 100 includes an apparatus 101 attached to an AFO frame 106. In an embodiment, the apparatus 101 includes a first frame portion or vertical frame 102 that is configured to attach to a shank portion 108 of the AFO frame 106. In this embodiment, the apparatus 101 also includes a second frame portion or horizontal frame 104 configured to attach to a foot portion 110 of the AFO frame 106. In one embodiment, upon attaching the apparatus 101 to the AFO frame 106, the horizontal frame 104 is configured to rotate relative to the vertical frame 102 about a rotational axis 112.

The use of the system 100 with respect to the ankle joint is now discussed. FIG. 1C is an image that illustrates an example of the system 100 of FIGS. 1A and 1B attached to an ankle of a subject, according to an embodiment. In one embodiment, the rotational axis 112 of the system 100 approximately aligns with a rotational axis 134 of an ankle of a subject wearing the AFO frame 106 and attached apparatus 101. In this example embodiment, the components of the apparatus 101 are oriented in a plane that is parallel to a plantar-dorsiflexion (PD) plane 136 such that the level of stiffness of the apparatus 101 about the rotational axis 112 is a level of stiffness in the PD plane 136 about the rotational axis 112. For purposes of this description, the PD plane 136 is the plane in which the foot rotates about the ankle rotational axis 134 during plantarflexion and dorsiflexion. Although FIGS. 1A through 1C depict the system 100 being used in conjunction with the ankle joint, the embodiments of the present invention are not limited to use with the ankle joint and can be configured to be utilized with other joints of the human body.

In an embodiment, the apparatus 101 includes a variable stiffness module configured to be attached between the shank portion 108 and foot portion 110 of the AFO frame 106. As appreciated by one of ordinary skill in the art, different conditions of movement of the AFO frame 106 (e.g., different gait phase, different speed, different incline, different surface, and/or different gait phase, etc.) may require different stiffness levels of the AFO frame 106. FIG. 1D is a diagram of a plurality of movement phases of a gait cycle 150 of a subject, according to an embodiment. The gait cycle 150 begins with an early stance 152 which includes a heel strike movement phase 154 and a mid-stance movement phase 156. The gait cycle 150 then proceeds to a late stance 158, which includes a heel off movement phase 160, and a toe off movement phase 162. The gait cycle 150 then proceeds to a swing 164 that includes an initial swing movement phase 166 and a terminal swing movement phase 168. In some embodiments, different stiffness levels are provided based on different movement phases of the gait cycle 150. In other embodiments, different stiffness levels are provided based on different speed, such as a running condition requiring a greater stiffness level in the leg prothesis 150 relative to a walking condition. In some embodiments, the apparatus 101 is calibrated by measuring the stiffness levels of the apparatus 101 at different known movement phases, known speeds, etc. In these embodiments, one or more sensors are used to measure the stiffness level of the apparatus 101. These stiffness levels are then stored in a memory of a controller 126 (FIG. 1A) of the system 100 and are utilized during operation of the system 100.

In some embodiments, the desired level of stiffness of the system 100 and corresponding parameters of the apparatus 101 stored in a memory of the controller 126 are determined in various ways. In one embodiment, the desired level of stiffness of the apparatus 101 is based on a measured parameter from a sensor (e.g. movement phase in the gait cycle 150, speed, etc.). FIG. 1E is an image that illustrate an example of a system 170 used to calibrate the stress of the apparatus 101 of the system 100 of FIG. 1A, according to an embodiment. The system 170 includes motion capture cameras 172 which are configured to gather image data indicating a position of reflective markers 176 mounted at various locations on the apparatus 101 and/or system 100. As a user walks while wearing the system 100, the user applies forces 174 which cause the various reflective markers 176 to change positions as the user walks through the movement phases of the gait cycle 150. The motion capture cameras 172 collect this image data (e.g., indicating the position of the reflective markers 176) and send this first data 182 to an inverse kinematics module 184. As the user walks through the gait cycle 150, force plates 178 on the ground measure the applied forces 174 and send this second data indicating the measured force data to a static optimization module 186. In these embodiments, the first data 182 (image data indicating the position of the reflective markers 176) and/or the second data 180 (force data) are combined at a AFO Torque and stiffness module 188 to determine the level of stiffness of the apparatus 101 at each movement phase of the gait cycle 150.

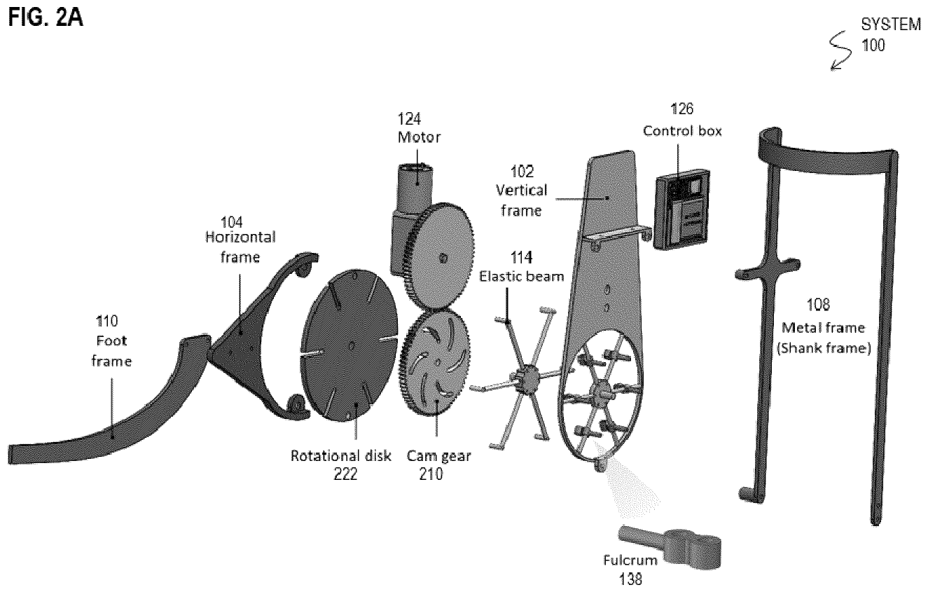

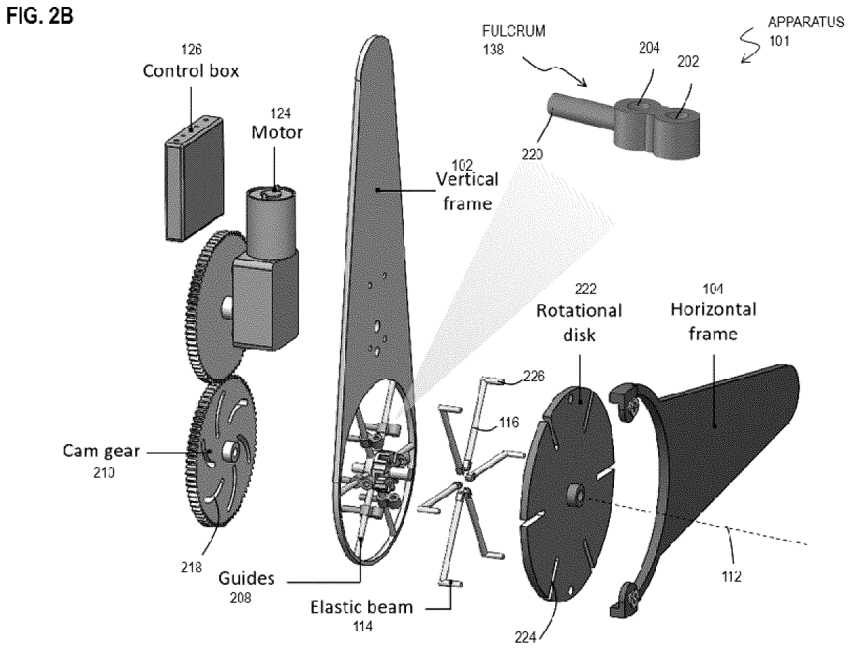

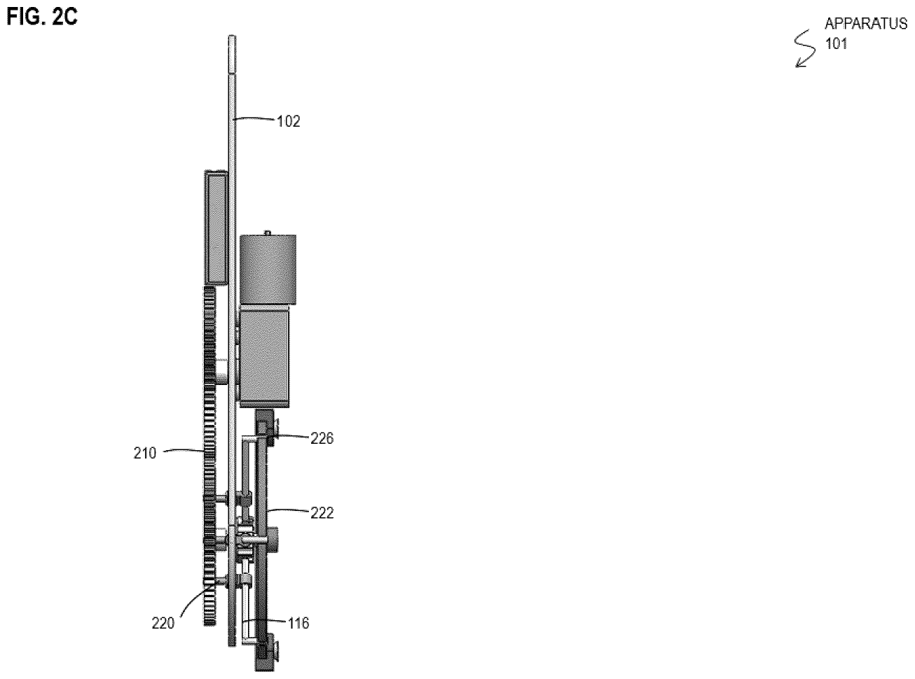

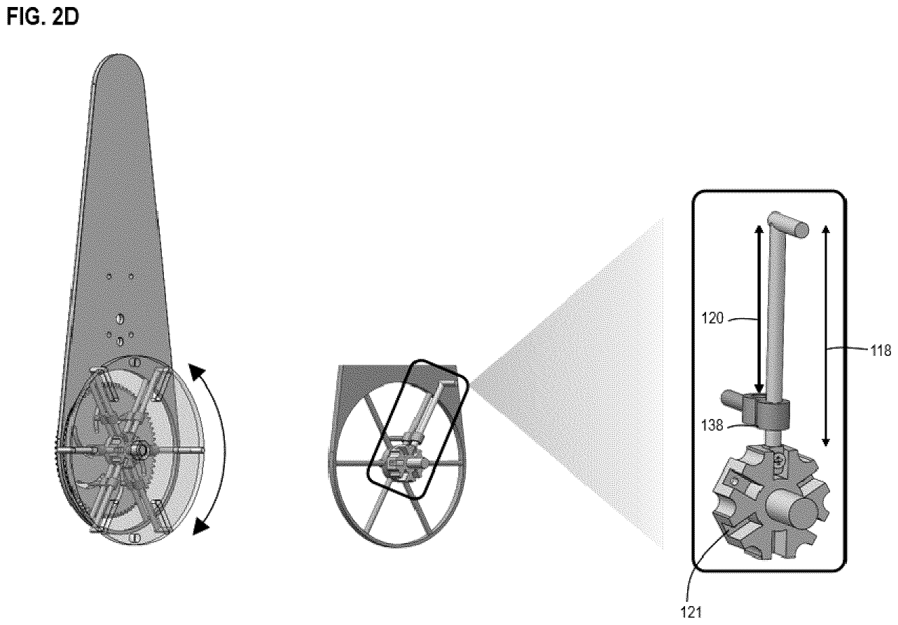

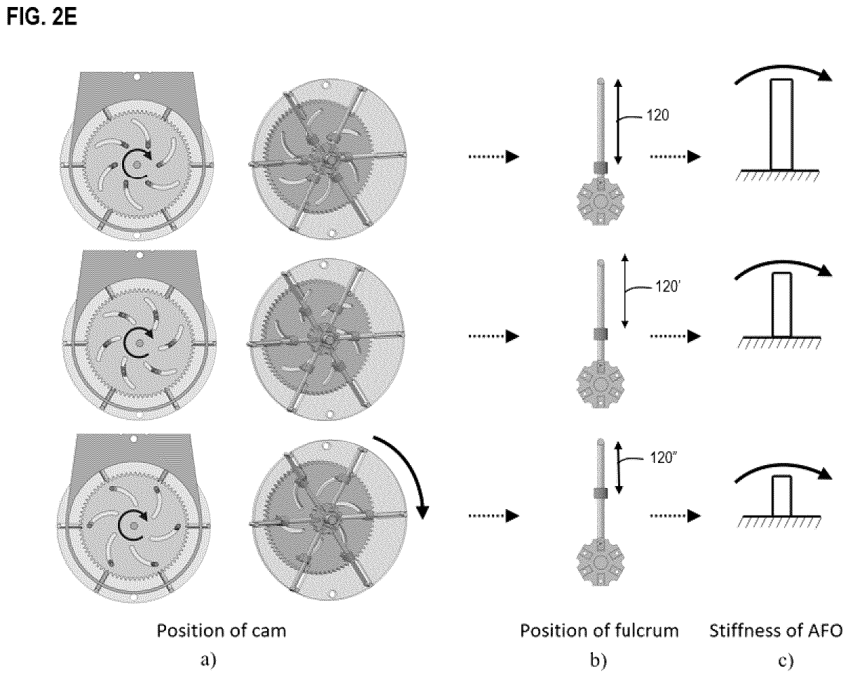

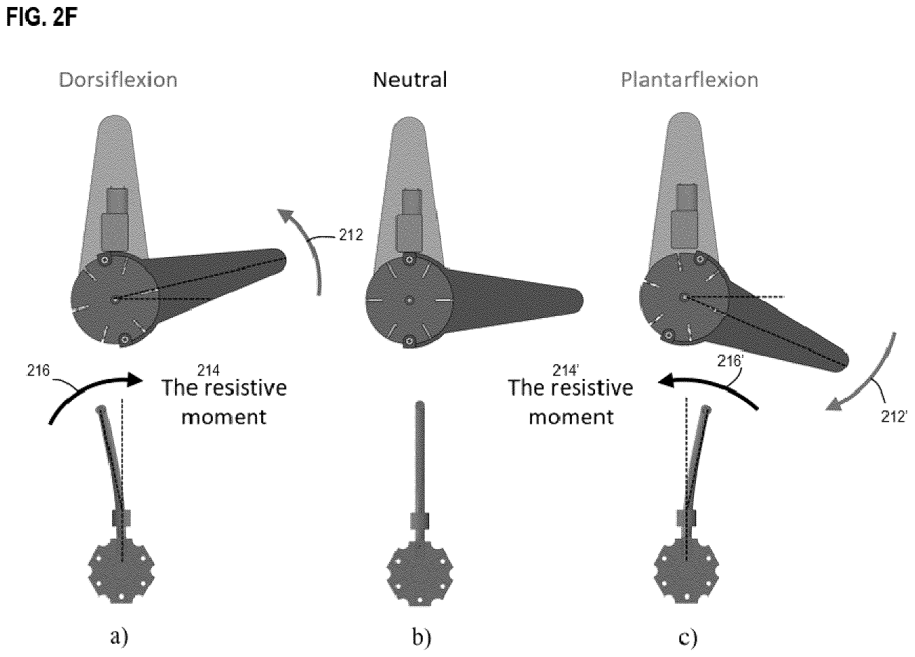

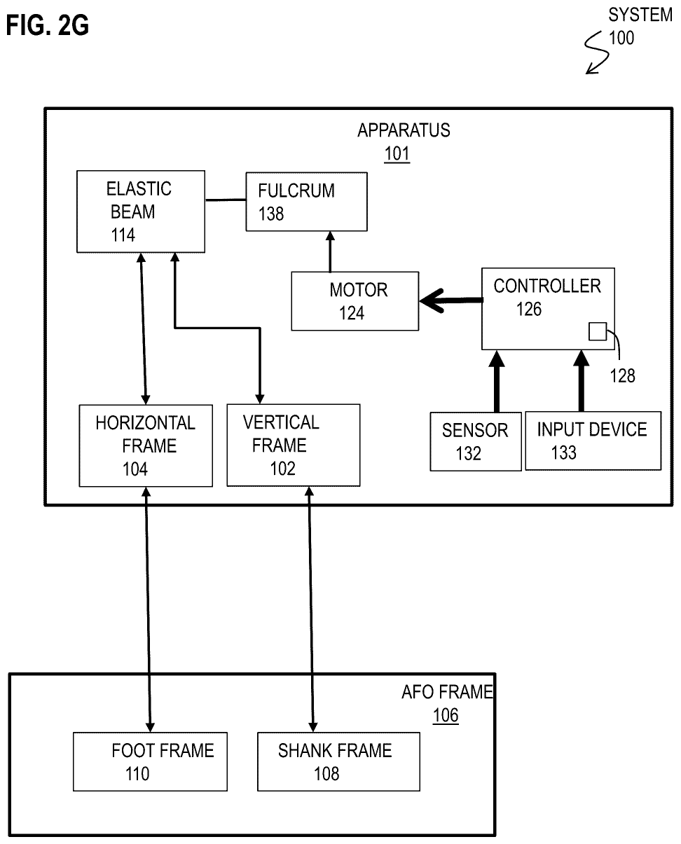

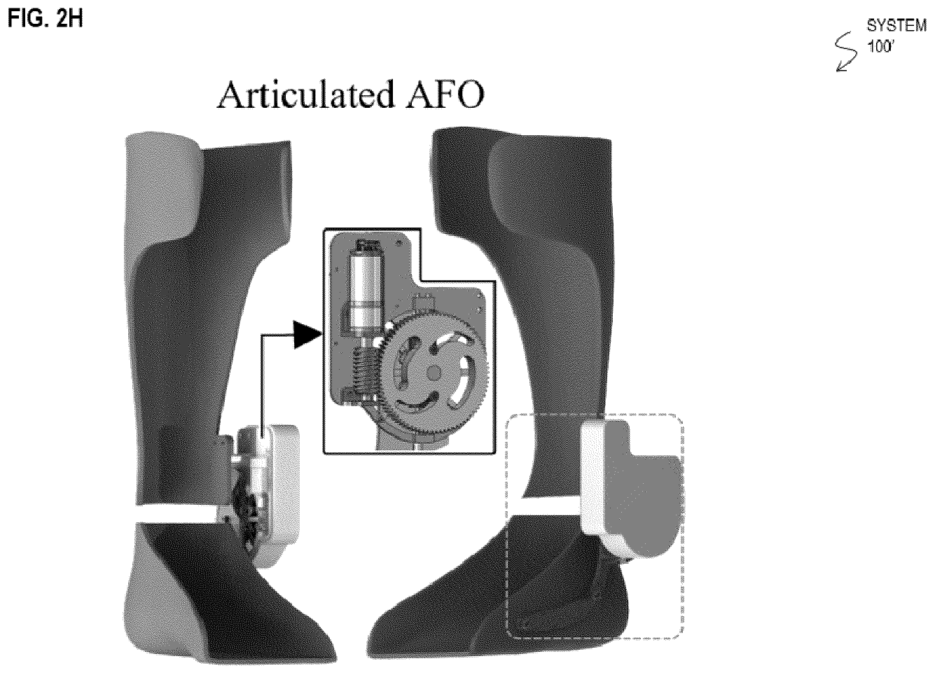

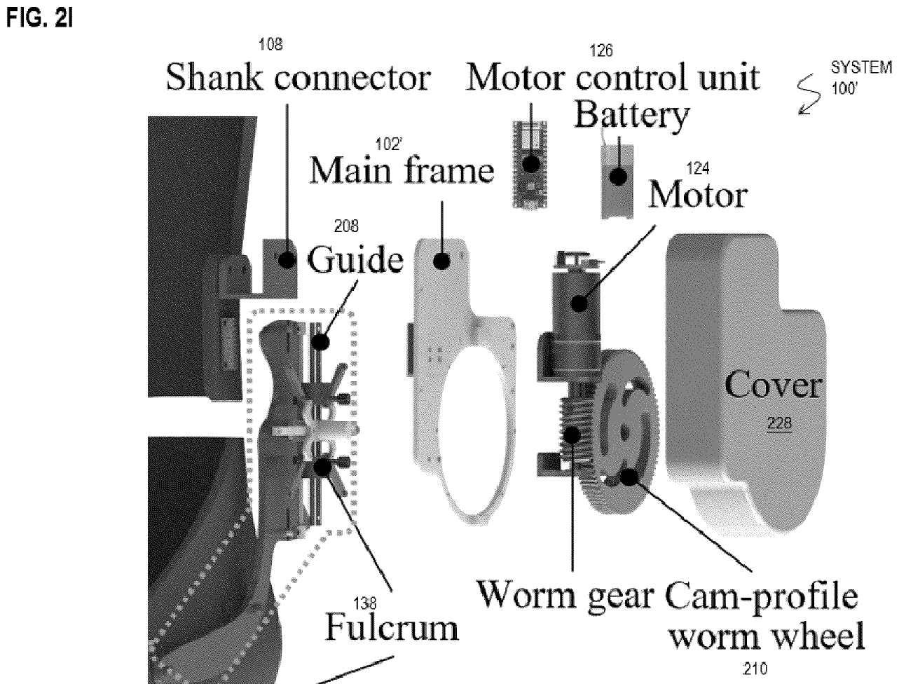

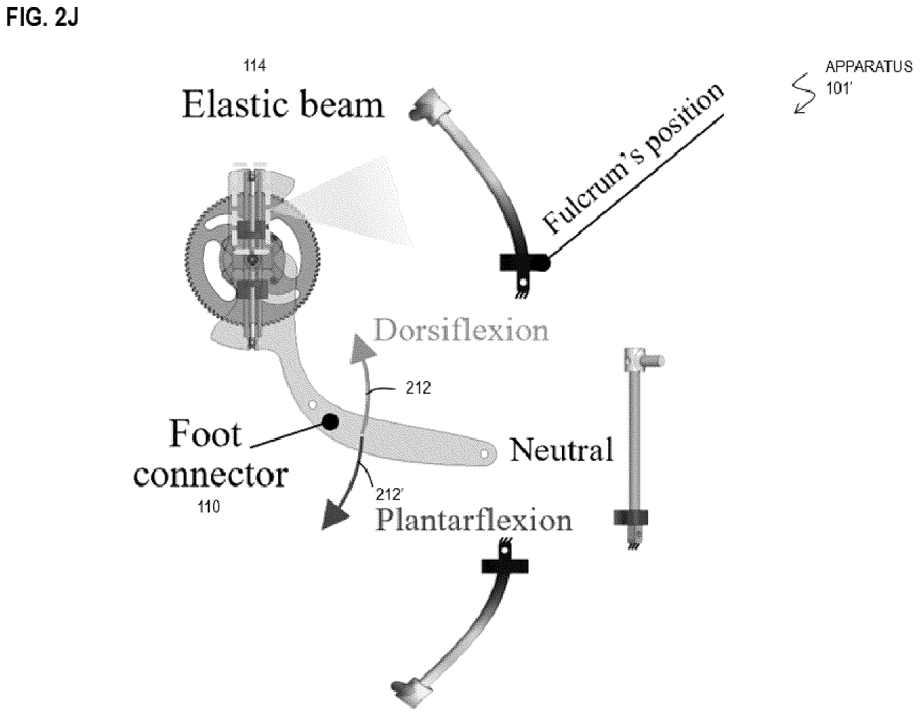

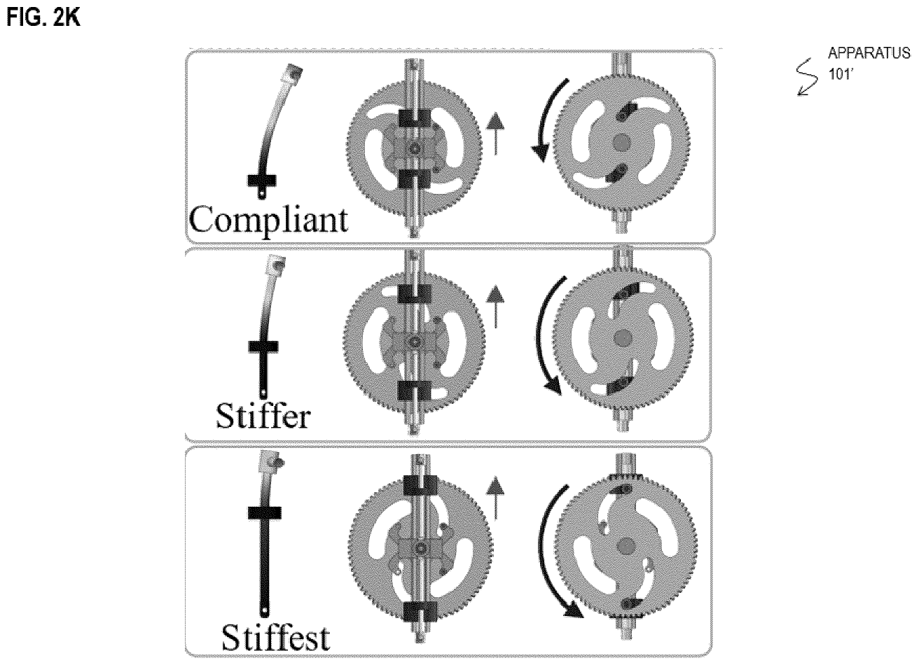

FIG. 2A is an image that illustrates an example of an exploded view of the system 100 of FIGS. 1A and 1B, according to an embodiment. FIG. 2B is an image that illustrates an example of an exploded view of the apparatus 101 of the system 100 of FIGS. 1A and 1B, according to an embodiment. As shown in FIGS. 2A and 2B, in an embodiment the apparatus 101 includes an elastic beam 114 including a plurality of segments 116 oriented with a first length 118 (FIG. 2D) in a radial direction that is orthogonal to the rotational axis 112. Additionally, in some embodiments the apparatus 101 includes a motor 124 operatively connected to the plurality of segments 116. In one embodiment, the motor 124 is configured to adjust an effective bend length 120 (FIG. 2D) of each of the plurality of segments 116 about the rotational axis 112. For purposes of this description, the “effective bend length” of the segments 116 is the length of the segments 116 that is capable of bending in the PD plane 136 when a rotational force in the PD plane 136 is exerted on the segments 116. As appreciated by one of ordinary skill in the art, the stiffness of the segments 116 (and hence the stiffness of the apparatus 101) in the PD plane 136 is inversely related to the effective bend length 120 such that increasing the effective bend length 120 of the segments 116 reduces the stiffness of the apparatus 101 in the PD plane 136 and decreasing the effective bend length 120 of the segments 116 increases the stiffness of the apparatus 101 in the PD plane 136. In this example embodiment, the effective bend length 120 is less than or equal to the first length 118 of the segments 116, which is shown in FIG. 2D.

Some additional structure of the apparatus 101 is now discussed, which is used to adjust the level of stiffness of the apparatus 101. FIG. 2C is an image that illustrates an example of a side view of the assembled system 100 of FIGS. 1A and 1C, according to an embodiment. FIG. 2D is an image that illustrates an example of the effective bend length 120 of the elastic beam segments 116 of FIGS. 2A and 2B based on the fulcrum 138 position, according to an embodiment. As shown in FIGS. 2A and 2B, the apparatus 101 includes a plurality of fulcrums 138. As shown in FIG. 2B, each fulcrum 138 defines a first opening 202 to slidably receive a respective segment 116 of the elastic beam 114. As shown in FIG. 2D, the effective bend length 120 of each segment 116 is defined between an outer radial tip of each segment 116 and the fulcrum 138. In this example embodiment, the motor 124 is configured to adjust the effective bend length 120 of each segment 116 based on adjustment of the position of the fulcrum 138 along each of the plurality of segments 116.

As shown in FIG. 2B, in an embodiment of the apparatus 101 the vertical frame 102 includes a plurality of guides 208 that are oriented in the radial direction (e.g. orthogonal to the rotational axis 112). As further shown in FIG. 2B, in one embodiment each fulcrum 138 further defines a second opening 204 to slidably receive a respective guide 208 of the plurality of guides 208 such that each fulcrum 138 is configured to slide along each guide 208 in the radial direction and is rotatably fixed to the vertical frame 102 about the rotational axis 112.

As further shown in FIG. 2B, the motor 124 is operatively connected to each fulcrum 138 such that the motor 124 is configured to adjust a position of each fulcrum 138 along each respective guide 208 which causes adjustment of each fulcrum 138 along each of the plurality of segments 116 and thus adjusts the effective bend length 120 of each segment 116. Hence, by adjusting the position of each fulcrum 138 along each respective guide 208, the motor 124 can effectively adjust the fulcrum 138 position along each segment 116 and thus adjust the stiffness of the apparatus 101 in the PD plane 136.

As shown in FIG. 2B, in some embodiments the motor 124 causes rotation of a first wheel with external teeth that matingly engages external teeth of a cam gear 210. This particular embodiment featuring the motor 124 will now be discussed herein. As shown in FIG. 2B, in one embodiment the cam gear 210 defines a plurality of arcuate slots 218. Each arcuate slot 218 is configured to receive a portion or end 220 of each fulcrum 138. In this example embodiment, the motor 124 is configured to rotate the cam gear 210 such that the portion or end 220 of each fulcrum 138 slides within the respective arcuate slot 218 which causes each fulcrum 138 to slide along each guide 208 in the radial direction to adjust the effective bend length 120 of each of the plurality of segments 116. Although this example embodiment discloses a motor 124 that is configured to adjust the effective bend length 120 via. rotation of the cam gear 210, in other embodiments any motor can be utilized which is capable of adjusting the effective bend length 120 whether such a motor employs rotational motion, linear motion or any other type of motion appreciated by one of skill in the art.

This adjustment of the effective bend length 120 of each segment 116 with the motor 124 is now discussed in more detail. FIG. 2E are images that illustrate an example of a position of the cam gear 210 (left side of FIG. 2E), a position of the fulcrum 138 (middle of FIG. 2E) and a stiffness of the AFO frame 106 (right side of FIG. 2E) of the system 100 of FIGS. 1A and 1B, according to an embodiment. As shown in the top row of FIG. 2E, in this embodiment the portion or end 220 of each fulcrum 138 is at an innermost radial position within each arcuate slot 218 of the cam gear 210. Consequently, as shown in the middle of the top row, the fulcrum 138 is positioned at a low position along the segment 116 and consequently the effective bend length 120 has a high value, leading to a low stiffness value of the AFO frame 106. As shown in the middle row of FIG. 2E, in this embodiment the portion or end 220 of each fulcrum 138 is at an intermediate radial position within each arcuate slot 218 of the cam gear 210. Consequently, as shown in the middle of the middle row, the fulcrum 138 is positioned at an intermediate position along the segment 116 and consequently the effective bend length 120′ has an intermediate value, leading to an intermediate stiffness value of the AFO frame 106. Finally, as shown in the bottom row of FIG. 2E, in this embodiment the portion or end 220 of each fulcrum 138 is at an outermost radial position within each arcuate slot 218 of the cam gear 210. Consequently, as shown in the middle of the bottom row, the fulcrum 138 is positioned at a high position along the segment 116 and consequently the effective bend length 120″ has a low value, leading to a high stiffness value of the AFO frame 106.

The operation of the system 100 will now be discussed, as it relates to various specific movement of the subject during the gait cycle 150. FIG. 2F are images that illustrate an example of a resistive moment 214 imparted by the elastic beam segments 116 of the apparatus 101 in response to dorsiflexion and plantarflexion, according to an embodiment. As shown in FIG. 2B, the apparatus 101 includes a rotational disk 222 operatively connected to the horizontal frame 104. The rotational disk 222 defines a plurality of radial slots 224, where each of the plurality of segments 116 of the elastic beam 114 include an outer radial tip 226 that extends in a direction orthogonal to the radial direction (or parallel to the rotational axis 112) to be received in a respective radial slot 224 of the plurality of slots 224. Consequently, upon rotation of the horizontal frame 104 relative to the vertical frame 102 during the gait cycle 150 in a first direction 212, the elastic beam 114 (via. the segments 116) is configured to impart a resistive moment 214 in a second direction 216 that is opposite to the first direction 212. The magnitude of the resistive moment 214 is based on the value of the level of stiffness of the apparatus 101 which is in turn based on the effective bend length 120 of the segments 116 of the elastic beam 114.

As shown in the left side of FIG. 2F, in one example embodiment during dorsiflexion (e.g. between mid stance movement phase 156 and heel off movement phase 160) the horizontal frame 104 rotates relative to the vertical frame 102 in a counterclockwise direction 212 (from the perspective of one viewing FIG. 2F) and the elastic beam 114 imparts the resistive moment 214 in a clockwise direction 216 in opposition to the dorsiflexion. Thus, this resistive moment 214 during dorsiflexion increases the stiffness of the system 100 and apparatus 101 during dorsiflexion. In an example embodiment, this increased stiffness during dorsiflexion may be introduced to address various physical issues (e.g. overly stiff calf muscle due to various neurological conditions, such as stroke or cerebral palsy, etc.). The inventors of the present invention surprisingly found that by increasing the stiffness of the system 100 and apparatus 101 in the PD plane 136, the overly stiff muscles during dorsiflexion for various subjects with these neurological conditions were improved or eliminated.

Similarly, as shown in the right side of FIG. 2F, in one example embodiment during plantarflexion (e.g. between toe off movement phase 162 and initial swing movement phase 166) the horizontal frame 104 rotates relative to the vertical frame 102 in a clockwise direction 212′ and the elastic beam 114 imparts the resistive moment 214 in a counter clockwise direction 216′ in opposition to the plantarflexion. Thus, this resistive moment 214 during plantarflexion increases the stiffness of the system 100 and apparatus 101 during plantarflexion. In some example embodiments, this resistive moment 214 during plantarflexion may be introduced to address various gait issues during plantarflexion (e.g. drop foot).

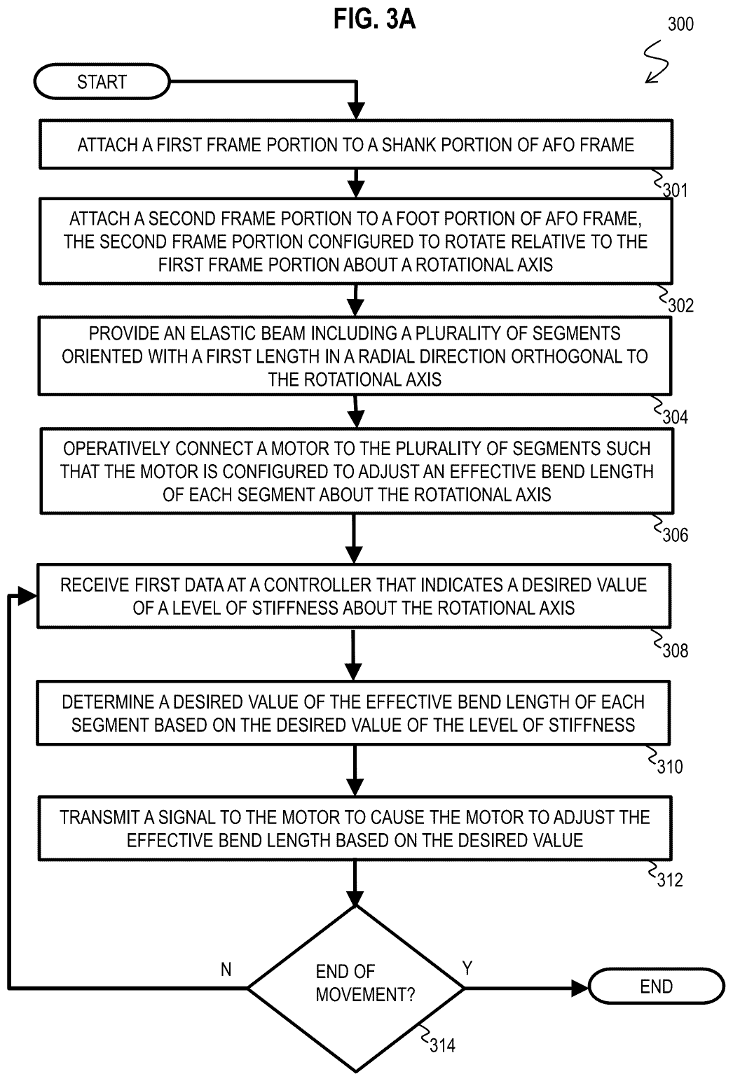

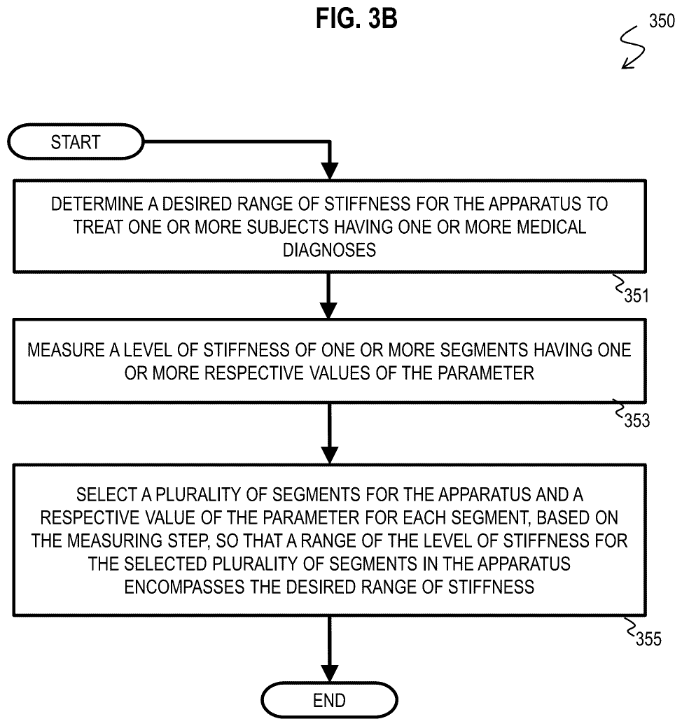

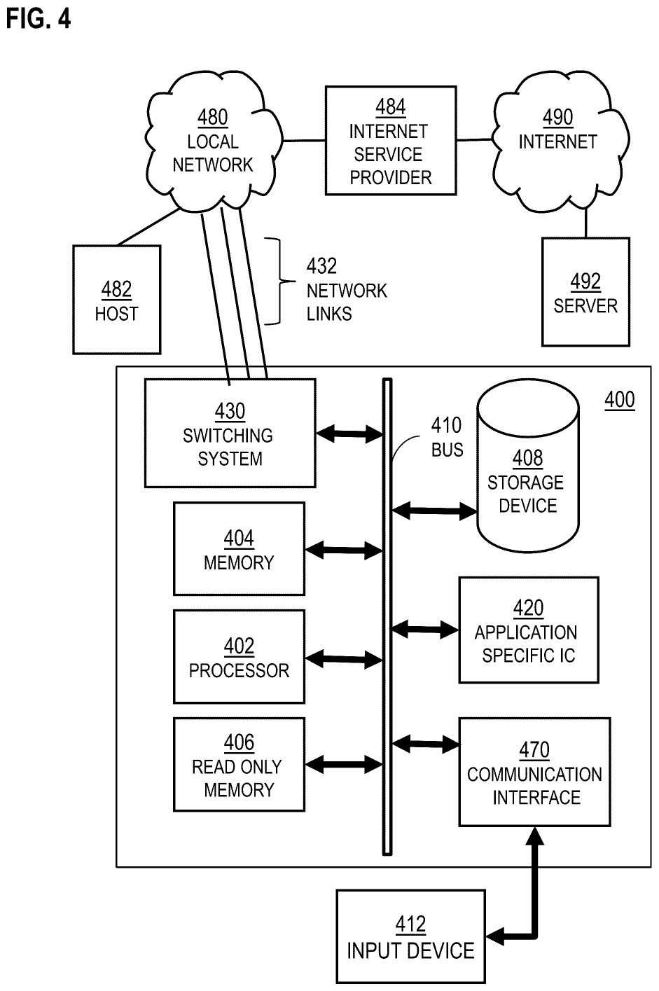

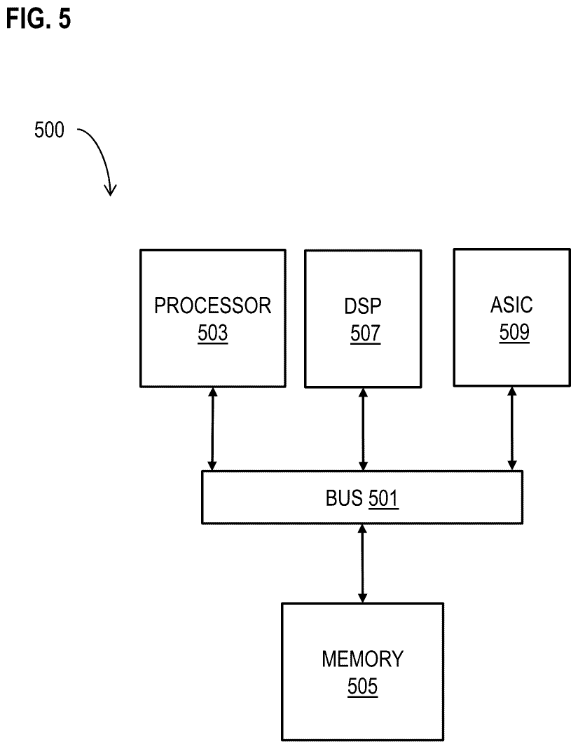

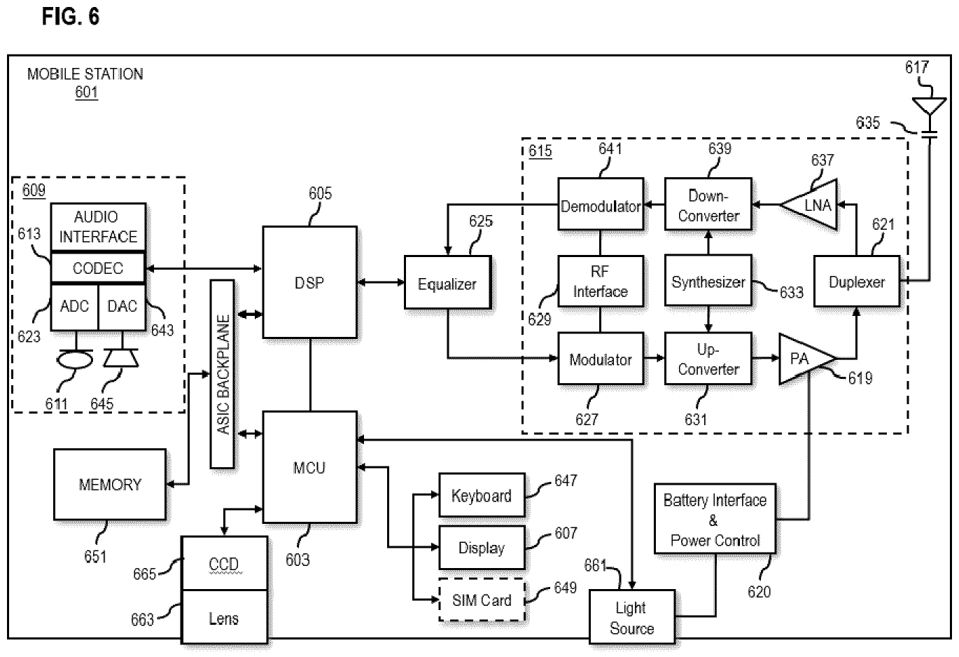

FIG. 2G is a block diagram that illustrates the components of the system 100 of FIG. 2A, according to an embodiment. As shown in FIG. 2G, in one embodiment the controller 126 is communicatively coupled with the motor 124. It should be noted that thick lines in FIG. 2G indicate a communication connection between components whereas less thick lines indicate a mechanical connection between components. As further shown in FIG. 2G, in one embodiment the controller 126 includes a memory 128 including one or more sequences of instructions. The memory 128 and the one or more sequences of instructions are configured to, with the controller 126 cause the apparatus 101 to perform one or more steps of the method 300 of FIG. 3. In an embodiment, the apparatus 101 of the system 100 includes the controller 126, such as a computer system 400 described below with reference to FIG. 4, a chip set 500 described below with reference to FIG. 5 or a mobile terminal 600 described below with reference to FIG. 6.

In one embodiment, the controller 126 is configured to receive first data that indicates a desired value of a level of stiffness of the apparatus 101 about the rotational axis 112. In one example embodiment, the apparatus 101 includes a sensor 132 that measures a value of a parameter of motion (e.g. speed, gait phase, etc.) and transmits this measured value of the parameter to the controller 126. The controller 126 then determines a desired value of the effective bend length 120 of the segments 116 based on the sensor data. In one example embodiment, the memory 128 of the controller 126 stores data that correlates the measured value of the parameter with the desired value of the effective bend length 120 (e.g. for higher speed of motion, a lower value of the effective bend length 120 to achieve a higher stiffness value; for lower speed of motion, a higher value of the effective bend length 120 to achieve a lower stiffness value, etc.). The controller 126 then transmits a signal to the motor 124 to cause the motor to move the fulcrums 138 by the necessary amount to achieve the desired value of the effective bend length 120 (e.g. via the cam gear 210) and hence the desired value of the level of stiffness.

Although FIGS. 2A through 2G depict one embodiment of the system 100 and apparatus 101, in other embodiments the system and apparatus may have one or more different design characteristics. FIGS. 2H through 2K are images that illustrate an example of a system 100′ and apparatus 101′ for providing variable stiffness in an AFO, according to an embodiment. The system 100′ and apparatus 101′ feature characteristics that are similar to the respective system 100 and apparatus 101, with the exception of the features discussed herein.

As shown in FIG. 21, the second frame portion or vertical frame 102′ has a different shape than the vertical frame 102 of FIG. 1B. Additionally, as shown in FIG. 21, the system 100′ may also include a cover 228 configured to cover the outside of the apparatus 101 that is attached to the vertical frame 102′ and horizontal frame 104. For example, the cover 228 may be sized to cover the rotational disk 222 and cam gear 210 which may advantageously provide a safety feature. In one example, the cover 228 may prevent injury due to contact with the rotational disk 222 during use of the system 100′.

As shown in FIG. 2K, the cam gear 210 may feature a different number of guides 208, fulcrums 138 and segments 116 than the apparatus 101 of FIGS. 2A through 2G. Although the depicted examples of the apparatus 101 of FIGS. 2A through 2G features six guides 208, six fulcrums 138 and six segments 116, in other embodiments less or more than six of each of these components may be provided in the apparatus 101. For example, as shown in FIG. 2K, the apparatus 101′ may feature two guides 208, two fulcrums 138 and two segments 116. In this example, the cam gear 210 may feature two arcuate slots 218 that are configured to receive the portions 220 of two fulcrums 138.

### 2. Method for Operating the System and Apparatus

FIG. 3A is a flow chart that illustrates an example method 300 for providing variable stiffness to the AFO of the system of FIGS. 1A and 1B, according to an embodiment. Although steps are depicted in FIG. 3A, and in the subsequent flowchart of FIG. 3B, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 301, the first frame portion or vertical frame 102 is attached to the shank portion 108 of the AFO frame 106.

In step 302, the second frame portion or horizontal frame 104 is attached to the foot portion 110 of the AFO frame 106. After performing step 302, the horizontal frame 104 is configured to rotate relative to the vertical frame 102 about the rotational axis 112.

In step 304, the elastic beam 114 is provided including the plurality of segments 116 oriented with the first length 118 in a radial direction that is orthogonal to the rotational axis 112. As shown in FIG. 2D, one end of the segments 116 is attached to a rotor 121 which is configured to rotate based on rotation of the cam gear 210.

In step 306, the motor 124 is operatively connected to the plurality of segments 116, such that the motor 124 is configured to adjust the effective bend length 120 of each of the plurality of segments 116 about the rotational axis 112. The effective bend length 120 is less than or equal to the first length 118.

In step 308, first data is received at the controller 126. In one embodiment, the first data indicates a desired value of a level of stiffness about the rotational axis 112. In one example embodiment, the first data is measured data from the sensor 132 that indicates a value of the parameter related to motion of the subject (e.g. speed data indicating a current speed, gait phase data indicating a current gait phase, etc.). In another example embodiment, the first data is input by an input device 133 (FIG. 2G) such as a keypad, a touchscreen, etc. where an operator of the system 100 manually inputs the desired value of the level of stiffness and/or manually inputs other data (e.g. current speed, current gait phase, etc.) that indicates the desired value of the level of stiffness. In some embodiments, the first data is based on the measured data from the modules 184, 186, 188, 190 of FIG. 1E. In other embodiments, the first data is based on manual observation of a subject walking by a medical professional who then inputs a desired value of the level of stiffness. The medical professional may then continue to observe the subject walking while the system 100 has the desired value of the level of stiffness and subsequently input a different value of the desired level of stiffness using the input device 133. This may continue until the medical professional observes the subject walking with ideal characteristics. In still other embodiments, a reinforcement learning algorithm or a machine learning algorithm can be used to determine the level of stiffness.

In step 310, the controller 126 determines a desired value of the effective bend length 120 of each of the plurality of segments 116 based on the desired value of the level of stiffness inputted in step 308. In these embodiments, the memory 128 of the controller 126 has calibration data stored therein that correlates the desired value of the level of stiffness with the desired value of the effective bend length 120. Thus, in step 310 the controller 126 retrieves from the memory 128 the desired value of the effective bend length 120 based on the desired value of the level of stiffness from step 308.

In step 312, the controller 126 transmits a signal the motor 124 to cause the motor 124 to adjust the effective bend length 120 of the segments 116 to the desired value. In a subsequent step, the motor 124 adjusts the effective bend length 120 to the desired value for each of the segments 116.

In step 314, the controller 126 determines whether the subject has finished moving (e.g. based on data received from the sensor 132 indicating no motion). If this determination is in the affirmative, the method 300 ends. Otherwise, the method 300 proceeds back to step 308.

In some embodiments, the method 300 includes a step of providing the plurality of fulcrums 138, where each fulcrum 138 defines the first opening 202. These embodiments of the method 300 also include a step of slidably receiving a respective segment 116 of the elastic beam 114 within the first opening 202 of each fulcrum 138. As shown in FIG. 2D, the effective bend length 120 is defined between an outer radial tip of each segment 116 and the fulcrum 138. Additionally, in these embodiments, the motor 124 adjusts the effective bend length 120 of the segments 116 based on adjusting the position of the fulcrum 138 along each of the plurality of segments 116.

In some embodiments, the method 300 includes slidably receiving a guide 208 of the plurality of guides 208 within a second opening 204 of each fulcrum 138 such that each fulcrum 138 is slidable along each guide 208 in the radial direction and each fulcrum 138 is rotatably fixed to the vertical frame 102 about the rotational axis 112.

In some embodiments, the operatively connecting 306 step includes operatively connecting the motor 124 to each fulcrum 138. In these embodiments, the motor 124 adjusts the position of the fulcrum 138 along each respective guide 208 which causes adjustment of each fulcrum 138 along each of the plurality of segments 116 and thus adjustment of the effective bend length 120 of each segment 116.

In some embodiments, the method 300 further includes providing the cam gear 210 defining the plurality of arcuate slots 218 and slidably receiving a portion (e.g. end 220) of each fulcrum 138 in each arcuate slot 218. In these embodiments, the motor 124 adjusts the effective bend length 120 by rotating the cam gear 210 such that the end 220 of each fulcrum 138 slides within each arcuate slot 218 and each fulcrum 138 slides along each guide 208 in the radial direction to adjust the effective bend length 120 of each of the plurality of segments 116.

In some embodiments, the method 300 further includes operatively connecting the rotational disk 222 to the horizontal frame 104, where the rotational disk 222 defines the plurality of radial slots 224. In these embodiments, the method 300 also includes a step of receiving the outer radial tip 226 of each segment 116 of the plurality of segments 116 of the elastic beam 114 in a respective radial slot 224 of the plurality of radial slots 224. Additionally, in these embodiments, the outer radial tip 226 of each segment 116 extends in a direction orthogonal to the radial direction or parallel to the rotational axis 112. In these embodiments, the method 300 also includes a step of imparting a resistive moment 214 by the elastic beam 114 in a second rotational direction 216 upon rotation of the horizontal frame 104 in a first rotational direction 212 that is opposite to the second rotational direction 216. A value of the resistive moment 214 is based on the desired value of the level of stiffness.

In some embodiments, the rotational axis 112 of the system 100 approximately aligns with a rotational axis 134 of an ankle of a subject wearing the AFO frame 106 with the attached horizontal frame 104 and vertical frame 102. In these embodiments, the plurality of segments 116 of the elastic beam 114 are in a plane that is about parallel to a plantar-dorsiflexion (PD) plane 136 such that the level of stiffness about the rotational axis 112 is a level of stiffness in the PD plane 136 about the rotational axis 112.

### 3. Method for Optimizing the Design of the System and Apparatus

A method is now disclosed for optimizing the design of the system 100 and apparatus 101. In one embodiment, the method involves optimizing the design of the apparatus 101 so that the apparatus 101 is capable of imparting a level of stiffness about the rotational axis 112 that is within a desired range of stiffness. In one example, this method may select one or more characteristics of the segments 116 of the elastic beam 114 (e.g. quantity, type of material, dimensions such as diameter, length, etc.).

FIG. 3B is a flow chart that illustrates an example method 350 for optimizing a value of a parameter of a component of the apparatus 101 of the system 100 of FIGS. 1A and 1B, according to an embodiment. In an initial step 351 of the method 350, a desired range of stiffness for the apparatus 101 and system 100 is first determined. This step 351 may involve selecting one or more medical diagnoses which the apparatus 101 and system 100 is designed to treat and determining the range of stiffness that would be required to treat subjects with these one or more medical diagnoses. For example, it was determined that an optimal stiffness for patients with reduced ankle push-off power is approximately 1.4 Nm/deg. Similarly, an optimal stiffness for patients with foot drop is about 6.36 Nm/deg. It was also determined that a minimum required stiffness of about 0.4-3.4 Nm/deg would be suitable for treating children and adults with varying gait patterns. Consequently, in one example, in step 351 it was determined that the desired range of stiffness for the apparatus 101 to treat subjects having one or more medical diagnoses is from about 0.4 Nm/deg to about 6.4 Nm/deg.

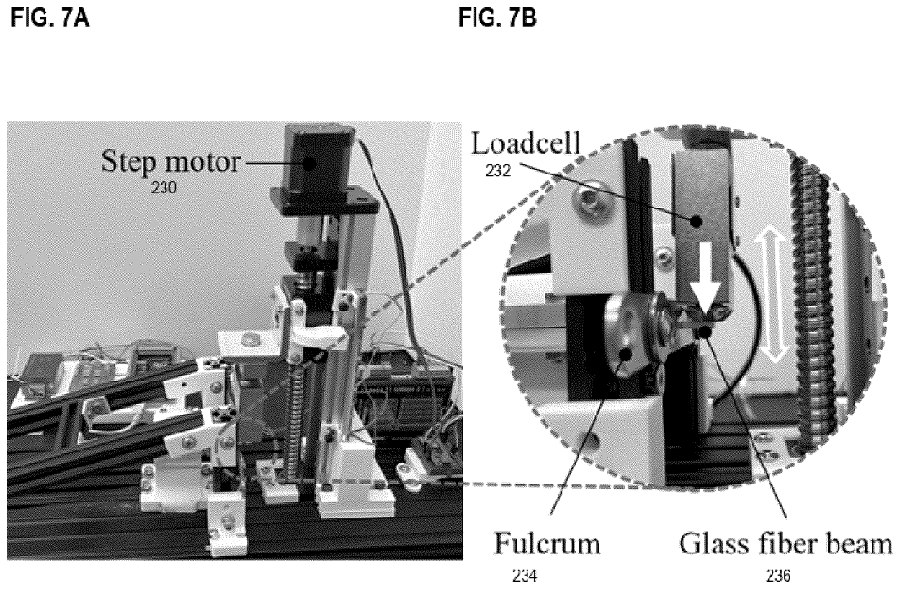

In step 353, a level of stiffness of one or more segments having one or more respective values of a parameter is measured. FIGS. 7A and 7B are images that illustrate an example of a step motor 230 and loadcell 232 used to optimize one or more design parameters of the system of FIGS. 1A through 1C, according to an embodiment. As shown in FIGS. 7A and 7B, a glass fiber beam 236 which may be a candidate for one of the segments 116 is positioned for the loadcell 232 to impose a force thereon (via. the step motor 230). A fulcrum 234 is provided to rotate the glass fiber beam 236 by a particular angle. The loadcell 232 and step motor 230 then determine an amount of work (force and distance) to move the glass fiber beam 236 back to a zero angle position. One or more graphs of the work versus angle data are generated for different glass fiber beams 236 with different parameters (e.g. diameter). These graphs will now be discussed herein.

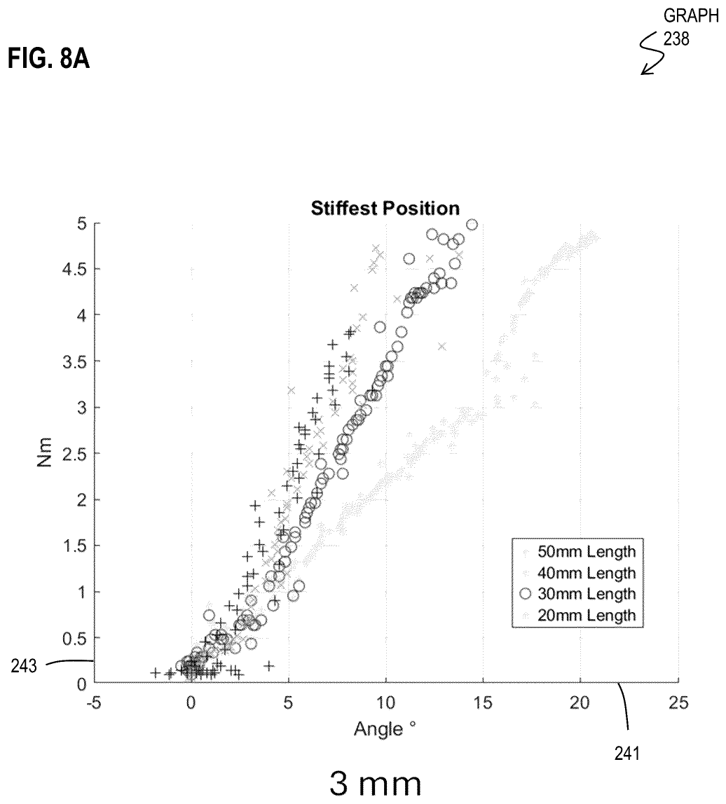

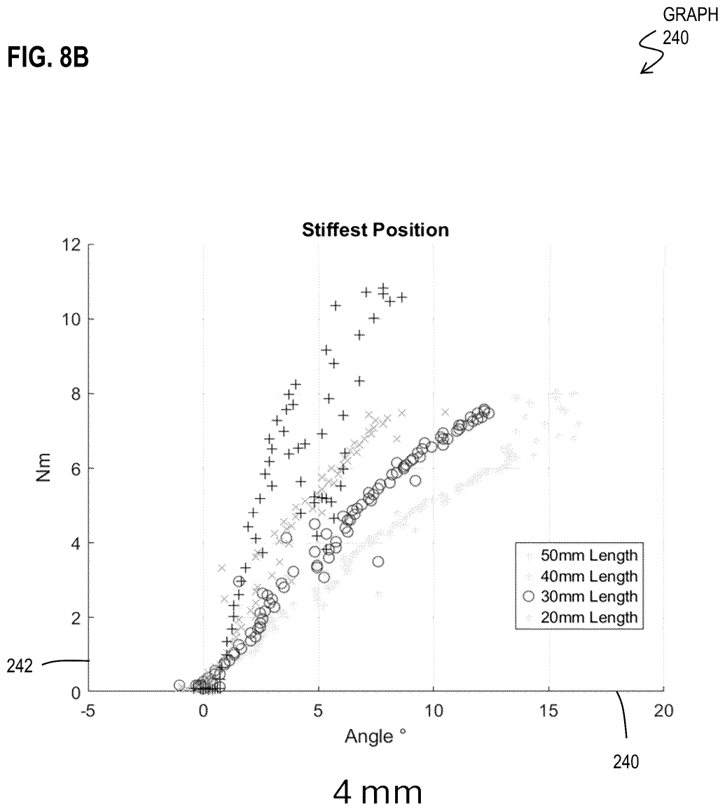

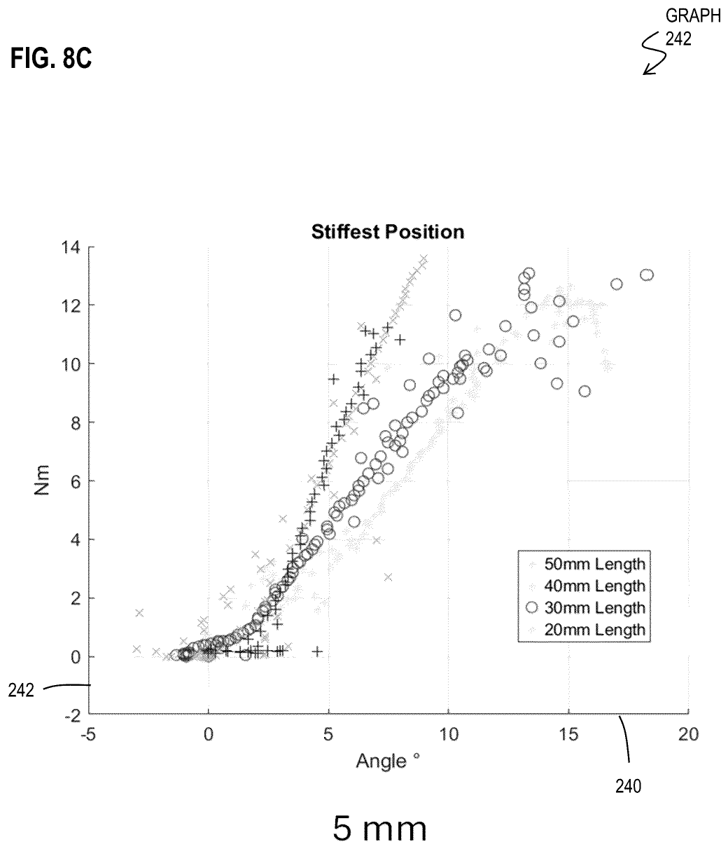

FIGS. 8A through 8C are graphs 238, 240, 242 illustrate an example of stiffness data captured using the step motor 230 and loadcell 232 of FIGS. 7A and 7B for elastic beam segments of different diameter, according to an embodiment. In one example, the graph 238 of FIG. 8A is captured using the glass fiber beam 236 with a first diameter (e.g. 3 mm), the graph 240 of FIG. 8B is captured using the glass fiber beam 236 with a second diameter (e.g. 4 mm) and the graph 242 of FIG. 8C is captured using the glass fiber beam 236 with a third diameter (e.g., 5 mm). The horizontal axis 241 of each graph is angle measured in degrees. The vertical axis 243 is work measured in units of force * distance (N*m). For purposes of this description, “stiffness” of the glass fiber beam 236 is a slope of the data in each graph or the rate of change of work (N*m) per degree of change in the angle. It should be noted that for each graph of FIGS. 8A through 8C, data is collected and depicted for a variety of lengths of the glass fiber beam 236 (e.g. corresponding to the effective bend length 120 of FIG. 2D).

It was recognized that the stiffness of the glass fiber beams 236 of FIGS. 8A and 8B (e.g. with the 3 mm and 4 mm diameters) did not provide sufficient stiffness, since the slope of the graphs 238, 240 did not encompass the desired range of stiffness of 0.4 Nm/deg to about 6.4 Nm/deg. However, it was found that the glass fiber beam 236 of FIG. 8C (e.g. with the 5 mm diameter) demonstrated a stiffness range from 0.2 to 5.8 Nm/° as the fulcrum position increased. This range exceeds the minimum required stiffness range of 0.4-3.4 Nm/°.

In step 355, a plurality of segments 116 for the elastic beam 114 are selected. In one example, in step 355 a quantity (e.g. five) segments 116 are determined. In another example, in step 355 the value of the parameter (e.g. diameter) is selected for each segment 116. In one example, four segments 116 having a diameter of 5 mm and one segment 116 having a diameter of 3 mm are selected in step 355. This selection of the diameters of the segments 116 is based on the data gathered in step 353. It was recognized that this combination of segments 116 provides a combined stiffness range from 0.41 Nm/deg to 6.97 Nm/deg, which advantageously encompasses the desired range of stiffness from step 351, which is from about 0.4 Nm/deg to about 6.4 Nm/deg.

### 4. Example Embodiments

In one embodiment, the goal of the system 100 and apparatus 101 design is to develop a simple, variable stiffness ankle-foot orthosis that can adjust the stiffness of the ankle joint during walking, as shown in FIG. 1C. In an embodiment, the apparatus 101 can be easily attached to the AFO frame 106 (e.g., any conventional AFO metal frames or plastic shells). In one example embodiment, the variable stiffness module of the apparatus 101 will be capable of adjusting the stiffness from about 1 Nm/° to about 4 Nm/°, but is light weight (˜1 kg) and has more energy efficiency to adjust the stiffness. In an example embodiment, the overall dimensions of the apparatus 101 is about 270 mm (length), about 285 mm (height) and about 40 mm (thick).

In one example embodiment, the system 100 includes vertical frame 102, horizontal frame 104, a motor 124, control box 126, cam gear 210 and elastic beam 114, as shown in FIG. 2A. The vertical frame 102 connects with the shank portion 108 that wrap around the calf and/or shank. In this example embodiment, the vertical frame 102 acts as a fixed link and will be in an upright, stable position. In this example embodiment, the vertical frame 102 has 6 stiff rods that act as guides 208 for the fulcrums 138. In this example embodiment, the rotational disk 222, which has a groove or radial slot 224 to not only hold the tip 220 of the fulcrum 138 but also to let the fulcrum 138 slide when it is shortened during walking, is linked to the horizontal frame 104. In an example embodiment, the horizontal frame 104 is then placed within the patient's shoes to allow movement of ankle dorsiflexion and plantarflexion. In this example embodiment, the motor 124 operates the cam gear 210 which has a profiled disc, making the fulcrum 138 to move up and down in a reciprocating motion. The position of the fulcrum 138 along the elastic beam 114 (e.g. segments 116 thereof) determines the stiffness of the apparatus 101.

In one example embodiment, the working principle of the proposed system 100 is designed based on a cantilever beam. In this example embodiment, the clastic beam 114 bears the force in its deformation which assists ankle dorsiflexion and plantarflexion movement by preventing opposite direction movement during the gait cycle 150.

In an example embodiment, FIG. 2F demonstrates the three states of the system 100: dorsiflexion, neutral, and plantarflexion. When dorsiflexion occurs (FIG. 2F, left side), the elastic beam 114 generates a resistive moment 214 that opposes the ankle motion, allowing the foot to return to the neutral position. During the upward motion of the foot, the resistive moment 214 will rise if plantarflexion occurs, as depicted in the right side of FIG. 2F. In one example embodiment, the rigidity of the system 100 can be modified by altering the type of elastic beam 114. The design allows for easy replacement of the elastic beam 114, making it portable and capable of providing various stiffness ranges that is suitable for children and adults. Additionally, a single system 100 can incorporate various combinations or types of beams 114 if needed.

In an example embodiment, the stiffness of the system 100 can be changed by turning the motor 124 while walking. In this example embodiment, FIG. 2E shows that the motor 124 will make the cam gear 210 turn while the patient walks. Thus, the fulcrums 138 will be in a different place depending on where the motor 124 is, and the system 100 will have a variable stiffness value.

### 5. Hardware Overview

FIG. 4 is a block diagram that illustrates a computer system 400 upon which an embodiment of the invention may be implemented. Computer system 400 includes a communication mechanism such as a bus 410 for passing information between other internal and external components of the computer system 400. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 400, or a portion thereof, constitutes a means for performing one or more steps of one or more methods described herein.

A sequence of binary digits constitutes digital data that is used to represent a number or code for a character. A bus 410 includes many parallel conductors of information so that information is transferred quickly among devices coupled to the bus 410. One or more processors 402 for processing information are coupled with the bus 410. A processor 402 performs a set of operations on information. The set of operations include bringing information in from the bus 410 and placing information on the bus 410. The set of operations also typically include comparing two or more units of information, shifting positions of units of information, and combining two or more units of information, such as by addition or multiplication. A sequence of operations to be executed by the processor 402 constitutes computer instructions.

Computer system 400 also includes a memory 404 coupled to bus 410. The memory 404, such as a random access memory (RAM) or other dynamic storage device, stores information including computer instructions. Dynamic memory allows information stored therein to be changed by the computer system 400. RAM allows a unit of information stored at a location called a memory address to be stored and retrieved independently of information at neighboring addresses. The memory 404 is also used by the processor 402 to store temporary values during execution of computer instructions. The computer system 400 also includes a read only memory (ROM) 406 or other static storage device coupled to the bus 410 for storing static information, including instructions, that is not changed by the computer system 400. Also coupled to bus 410 is a non-volatile (persistent) storage device 408, such as a magnetic disk or optical disk, for storing information, including instructions, that persists even when the computer system 400 is turned off or otherwise loses power.

Information, including instructions, is provided to the bus 410 for use by the processor from an external input device 412, such as a keyboard containing alphanumeric keys operated by a human user, or a sensor. A sensor detects conditions in its vicinity and transforms those detections into signals compatible with the signals used to represent information in computer system 400. Other external devices coupled to bus 410, used primarily for interacting with humans, include a display device 414, such as a cathode ray tube (CRT) or a liquid crystal display (LCD), for presenting images, and a pointing device 416, such as a mouse or a trackball or cursor direction keys, for controlling a position of a small cursor image presented on the display 414 and issuing commands associated with graphical elements presented on the display 414.

In the illustrated embodiment, special purpose hardware, such as an application specific integrated circuit (IC) 420, is coupled to bus 410. The special purpose hardware is configured to perform operations not performed by processor 402 quickly enough for special purposes. Examples of application specific ICs include graphics accelerator cards for generating images for display 414, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 400 also includes one or more instances of a communications interface 470 coupled to bus 410. Communication interface 470 provides a two-way communication coupling to a variety of external devices that operate with their own processors, such as printers, scanners and external disks. In general the coupling is with a network link 478 that is connected to a local network 480 to which a variety of external devices with their own processors are connected. For example, communication interface 470 may be a parallel port or a serial port or a universal serial bus (USB) port on a personal computer. In some embodiments, communications interface 470 is an integrated services digital network (ISDN) card or a digital subscriber line (DSL) card or a telephone modem that provides an information communication connection to a corresponding type of telephone line. In some embodiments, a communication interface 470 is a cable modem that converts signals on bus 410 into signals for a communication connection over a coaxial cable or into optical signals for a communication connection over a fiber optic cable. As another example, communications interface 470 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 470 sends and receives electrical, acoustic or electromagnetic signals, including infrared and optical signals, that carry information streams, such as digital data.

The term computer-readable medium is used herein to refer to any medium that participates in providing information to processor 402, including instructions for execution. Such a medium may take many forms, including, but not limited to, non-volatile media, volatile media and transmission media. Non-volatile media include, for example, optical or magnetic disks, such as storage device 408. Volatile media include, for example, dynamic memory 404. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 402, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 402, except for carrier waves and other signals.

Logic encoded in one or more tangible media includes one or both of processor instructions on a computer-readable storage media and special purpose hardware, such as ASIC *420.

Network link 478 typically provides information communication through one or more networks to other devices that use or process the information. For example, network link 478 may provide a connection through local network 480 to a host computer 482 or to equipment 484 operated by an Internet Service Provider (ISP). ISP equipment 484 in turn provides data communication services through the public, world-wide packet-switching communication network of networks now commonly referred to as the Internet 490. A computer called a server 492 connected to the Internet provides a service in response to information received over the Internet. For example, server 492 provides information representing video data for presentation at display 414.

The invention is related to the use of computer system 400 for implementing the techniques described herein. According to one embodiment of the invention, those techniques are performed by computer system 400 in response to processor 402 executing one or more sequences of one or more instructions contained in memory 404. Such instructions, also called software and program code, may be read into memory 404 from another computer-readable medium such as storage device 408. Execution of the sequences of instructions contained in memory 404 causes processor 402 to perform the method steps described herein. In alternative embodiments, hardware, such as application specific integrated circuit 420, may be used in place of or in combination with software to implement the invention. Thus, embodiments of the invention are not limited to any specific combination of hardware and software.

The signals transmitted over network link 478 and other networks through communications interface 470, carry information to and from computer system 400. Computer system 400 can send and receive information, including program code, through the networks 480, 490 among others, through network link 478 and communications interface 470. In an example using the Internet 490, a server 492 transmits program code for a particular application, requested by a message sent from computer 400, through Internet 490, ISP equipment 484, local network 480 and communications interface 470. The received code may be executed by processor 402 as it is received, or may be stored in storage device 408 or other non-volatile storage for later execution, or both. In this manner, computer system 400 may obtain application program code in the form of a signal on a carrier wave.

Various forms of computer readable media may be involved in carrying one or more sequence of instructions or data or both to processor 402 for execution. For example, instructions and data may initially be carried on a magnetic disk of a remote computer such as host 482. The remote computer loads the instructions and data into its dynamic memory and sends the instructions and data over a telephone line using a modem. A modem local to the computer system 400 receives the instructions and data on a telephone line and uses an infra-red transmitter to convert the instructions and data to a signal on an infra-red a carrier wave serving as the network link 478. An infrared detector serving as communications interface 470 receives the instructions and data carried in the infrared signal and places information representing the instructions and data onto bus 410. Bus 410 carries the information to memory 404 from which processor 402 retrieves and executes the instructions using some of the data sent with the instructions. The instructions and data received in memory 404 may optionally be stored on storage device 408, either before or after execution by the processor 402.

FIG. 5 illustrates a chip set 500 upon which an embodiment of the invention may be implemented. Chip set 500 is programmed to perform one or more steps of a method described herein and includes, for instance, the processor and memory components described with respect to FIG. *4 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 500, or a portion thereof, constitutes a means for performing one or more steps of a method described herein.

In one embodiment, the chip set 500 includes a communication mechanism such as a bus 501 for passing information among the components of the chip set 500. A processor 503 has connectivity to the bus 501 to execute instructions and process information stored in, for example, a memory 505. The processor 503 may include one or more processing cores with each core configured to perform independently. A multi-core processor enables multiprocessing within a single physical package. Examples of a multi-core processor include two, four, eight, or greater numbers of processing cores. Alternatively or in addition, the processor 503 may include one or more microprocessors configured in tandem via the bus 501 to enable independent execution of instructions, pipelining, and multithreading. The processor 503 may also be accompanied with one or more specialized components to perform certain processing functions and tasks such as one or more digital signal processors (DSP) 507, or one or more application-specific integrated circuits (ASIC) 509. A DSP 507 typically is configured to process real-world signals (e.g., sound) in real time independently of the processor 503. Similarly, an ASIC 509 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 503 and accompanying components have connectivity to the memory 505 via the bus 501. The memory 505 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 505 also stores the data associated with or generated by the execution of one or more steps of the methods described herein.

FIG. 6 is a diagram of exemplary components of a mobile terminal 600 (e.g., cell phone handset) for communications, which is capable of operating in the system of FIG. 2C, according to one embodiment. In some embodiments, mobile terminal 601, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 603, a Digital Signal Processor (DSP) 605, and a receiver/transmitter unit including a microphone gain control unit and a speaker gain control unit. A main display unit 607 provides a display to the user in support of various applications and mobile terminal functions that perform or support the steps as described herein. The display 607 includes display circuitry configured to display at least a portion of a user interface of the mobile terminal (e.g., mobile telephone). Additionally, the display 607 and display circuitry are configured to facilitate user control of at least some functions of the mobile terminal. An audio function circuitry 609 includes a microphone 611 and microphone amplifier that amplifies the speech signal output from the microphone 611. The amplified speech signal output from the microphone 611 is fed to a coder/decoder (CODEC) 613.

A radio section 615 amplifies power and converts frequency in order to communicate with a base station, which is included in a mobile communication system, via antenna 617. The power amplifier (PA) 619 and the transmitter/modulation circuitry are operationally responsive to the MCU 603, with an output from the PA 619 coupled to the duplexer 621 or circulator or antenna switch, as known in the art. The PA 619 also couples to a battery interface and power control unit 620.

In use, a user of mobile terminal 601 speaks into the microphone 611 and his or her voice along with any detected background noise is converted into an analog voltage. The analog voltage is then converted into a digital signal through the Analog to Digital Converter (ADC) 623. The control unit 603 routes the digital signal into the DSP 605 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 625 for compensation of any frequency-dependent impairments that occur during transmission though the air such as phase and amplitude distortion. After equalizing the bit stream, the modulator 627 combines the signal with a RF signal generated in the RF interface 629. The modulator 627 generates a sine wave by way of frequency or phase modulation. In order to prepare the signal for transmission, an up-converter 631 combines the sine wave output from the modulator 627 with another sine wave generated by a synthesizer 633 to achieve the desired frequency of transmission. The signal is then sent through a PA 619 to increase the signal to an appropriate power level. In practical systems, the PA 619 acts as a variable gain amplifier whose gain is controlled by the DSP 605 from information received from a network base station. The signal is then filtered within the duplexer 621 and optionally sent to an antenna coupler 635 to match impedances to provide maximum power transfer. Finally, the signal is transmitted via antenna 617 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 601 are received via antenna 617 and immediately amplified by a low noise amplifier (LNA) 637. A down-converter 639 lowers the carrier frequency while the demodulator 641 strips away the RF leaving only a digital bit stream. The signal then goes through the equalizer 625 and is processed by the DSP 605. A Digital to Analog Converter (DAC) 643 converts the signal and the resulting output is transmitted to the user through the speaker 645, all under control of a Main Control Unit (MCU) 603 which can be implemented as a Central Processing Unit (CPU) (not shown).

The MCU 603 receives various signals including input signals from the keyboard 647. The keyboard 647 and/or the MCU 603 in combination with other user input components (e.g., the microphone 611) comprise a user interface circuitry for managing user input. The MCU 603 runs a user interface software to facilitate user control of at least some functions of the mobile terminal 601 as described herein. The MCU 603 also delivers a display command and a switch command to the display 607 and to the speech output switching controller, respectively. Further, the MCU 603 exchanges information with the DSP 605 and can access an optionally incorporated SIM card 649 and a memory 651. In addition, the MCU 603 executes various control functions required of the terminal. The DSP 605 may, depending upon the implementation, perform any of a variety of conventional digital processing functions on the voice signals. Additionally, DSP 605 determines the background noise level of the local environment from the signals detected by microphone 611 and sets the gain of microphone 611 to a level selected to compensate for the natural tendency of the user of the mobile terminal 601.

The CODEC 613 includes the ADC 623 and DAC 643. The memory 651 stores various data including call incoming tone data and is capable of storing other data including music data received via, e.g., the global Internet. The software module could reside in RAM memory, flash memory, registers, or any other form of writable storage medium known in the art. The memory device 651 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 649 carries, for instance, important information, such as the cellular phone number, the carrier supplying service, subscription details, and security information. The SIM card 649 serves primarily to identify the mobile terminal 601 on a radio network. The card 649 also contains a memory for storing a personal telephone number registry, text messages, and user specific mobile terminal settings.

In some embodiments, the mobile terminal 601 includes a digital camera comprising an array of optical detectors, such as charge coupled device (CCD) array 665. The output of the array is image data that is transferred to the MCU for further processing or storage in the memory 651 or both. In the illustrated embodiment, the light impinges on the optical array through a lens 663, such as a pin-hole lens or a material lens made of an optical grade glass or plastic material. In the illustrated embodiment, the mobile terminal 601 includes a light source 661, such as a LED to illuminate a subject for capture by the optical array, e.g., CCD 665. The light source is powered by the battery interface and power control module 620 and controlled by the MCU 603 based on instructions stored or loaded into the MCU 603.

### 6. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 7. REFERENCES

[1] Aboutorabi A, Arazpour M, Bani M A, Saeedi H, Head J S, “Efficacy of ankle foot orthoses types on walking in children with cerebral palsy: A systematic review,” Annals of physical and rehabilitation medicine, vol. 60, no. 6, pp. 393-402, 2017.
[2] Wingstrand M, Hägglund G, Rodby-Bousquet E, “Ankle-foot orthoses in children with cerebral palsy: a cross sectional population based study of 2200 children,” BMC musculoskeletal disorders, vol. 15, no. 1, pp. 1-7, 2014.
[3] J. P. Betancourt, P. Eleeh, S. Stark and N. B. Jain, “Impact of ankle-foot orthosis on gait efficiency in ambulatory children with cerebral palsy: a systematic review and meta-analysis,” American journal of physical medicine & rehabilitation, vol. 98, no. 9, pp. 759-770, 2019.
[4] C. Morris and D. Condie, “Recent developments in healthcare for cerebral palsy: Implications and opportunities for orthotics,” Copenhagen: International Society for Prosthetics and Orthotics, pp. 6-18, 2009.
[5] S. H. Collins, M. B. Wiggin and G. S. Sawicki, “Reducing the energy cost of human walking using an unpowered exoskeleton,” Nature, vol. 522, no. 7555, pp. 212-215, 2015.
[6] W. A. El-Hakiem Abd El-Nabie, H. G. Abd El Aziz and S. Elshennawy, “Effect of walking aids and foot orthoses on energy expenditure in children with cerebral palsy: a systematic review,” Bulletin of Faculty of Physical Therapy, vol. 24, pp. 99-112, 2019.
[7] C. Bayón, M. van Hoorn, A. Barrientos, E. Rocon, J. P. Trost and E. H. van Asseldonk, “Perspectives on ankle-foot technology for improving gait performance of children with Cerebral Palsy in daily-life: requirements, needs and wishes,” Journal of neuroengineering and rehabilitation, vol. 20, no. 1, p. 44, 2023.
[8] Totah D, Menon M, Jones-Hershinow C, Barton K, Gates D H, “The impact of ankle-foot orthosis stiffness on gait: A systematic literature review,” Gait Posture, vol. 69, p. 101-111, 2019.
[9] Y. L. Kerkum, A. I. Buizer, J. C. Van Den Noort, J. G. Becher, J. Harlaar and M. A. Brehm, “The effects of varying ankle foot orthosis stiffness on gait in children with spastic cerebral palsy who walk with excessive knee flexion,” PloS one, vol. 10, no. 11, p. e0142878, 2015.
[10] Bayón C, Hoorn M V, Barrientos A, Rocon E, Trost J P, Asseldonk E H, “Perspectives on ankle-foot technology for improving gait performance of children with cerebral palsy in daily-life: Requirements, needs and wishes,” J. Neuroeng. Rehabil., vol. 20, 2023.
[11] Waterval N F, Brehm M A, Harlaar J, Nollet F. Waterval, “Individual stiffness optimization of dorsal leaf spring ankle-foot orthoses in people with calf muscle weakness is superior to standard bodyweight-based recommendations,” J. Neuroeng. Rehabil, vol. 18, 2021.
[12] C. Zhou, Z. Yang, K. Li and X. Ye, “Research and development of ankle-foot orthoses: A review,” Sensors, vol. 22, no. 17, p. 6596, 2022.
[13] T. Jiang and W. Durfee, “Bench testing of a tunable ankle-foot orthosis with adjustable stiffness and neutral angle,” In Frontiers in Biomedical Devices, vol. 86731, p. V001T04A002, 2023.
[14] R. Anderson, “Design of A Variable-Stiffness Ankle-Foot Orthosis Emulator,” Doctoral dissertation, University of Minnesota, 2020.
[15] Y. Shao, W. Zhang, Y. Su and X. Ding, “Design and optimisation of load-adaptive actuator with variable stiffness for compact ankle exoskeleton,” Mechanism and Machine Theory, vol. 161, p. 104323, 2021.
[16] M. G. Mataee, M. T. Andani and M. Elahinia, “Adaptive ankle-foot orthoses based on superelasticity of shape memory alloys,” Journal of intelligent material systems and structures, vol. 26, no. 6, pp. 639-651, 2015.
[17] D. Totah, K. Barton and D. H. Gates, “The effect of rotational speed on ankle-foot orthosis properties,” Journal of biomechanics, vol. 123, p. 110483, 2021.
[18] H. A. Quraishi, M. K. Shepherd, L. McManus, J. Harlaar, D. H. Plettenburg and E. J. Rouse, “A passive mechanism for decoupling energy storage and return in ankle-foot prostheses: A case study in recycling collision energy,” Wearable Technologies, vol. 2, p. e9, 2021.
[19] M. D. Geil, “Energy loss and stiffness properties of dynamic elastic response prosthetic feet,” JPO: Journal of Prosthetics and Orthotics, vol. 13, no. 3, pp. 70-73, 2001.
[20] DeBoer B, Hosseini A, Rossa C, “A discrete non-linear series elastic actuator for active ankle-foot orthoses,” IEEE Robotics and Automation Letters, 2022.
[21] Kim S J, Na Y, Lee D Y, Chang H, Kim J, “Pneumatic AFO powered by a miniature custom compressor for drop foot correction,” IEEE Transactions on Neural Systems and Rehabilitation Engineering, 2020.
[22] Wang J, Shu J, Su Y, Hu C, Yeung L F, Li Z, Tong R K, “portable AFO solution for pneumatic actuator with cable tendon mechanism to assist ankle dorsiflexion,” Frontiers in Bioengineering and Biotechnology, 2023.
[23] Van Crey N, Cavallin M, Shepherd M, Rouse E J, “Design of a Quasi-Passive Ankle-Foot Orthosis with Customizable, Variable Stiffness,” In2023 International Conference on Rehabilitation Robotics (ICORR).IEEE, pp. 1-6, 2023.
[24] D. Totah, M. Menon, C. Jones-Hershinow, K. Barton and D. H. Gates, “The impact of ankle-foot orthosis stiffness on gait: A systematic literature review,” Gait & posture, vol. 69, pp. 101-111, 2019.

What is claimed is:

1. An apparatus comprising:
a first frame portion configured to attach to a shank portion of an ankle-foot orthosis (AFO) frame;
a second frame portion configured to attach to a foot portion of the AFO frame, wherein the second frame portion is configured to rotate relative to the first frame portion about a rotational axis when attached to the AFO frame;
an elastic beam comprising a plurality of segments oriented with a first length in a radial direction that is orthogonal to the rotational axis;
a motor operatively connected to the plurality of segments, such that the motor is configured to adjust an effective bend length of each of the plurality of segments about the rotational axis, wherein the effective bend length is less than or equal to the first length;
a controller communicatively coupled with the motor; and
at least one memory including one or more sequences of instructions,
the at least one memory and the one or more sequences of instructions configured to, with the at least one processor, cause the apparatus to perform at least the following,
receive first data indicating a desired value of a level of stiffness of the apparatus about the rotational axis,
determine a desired value of the effective bend length of each of the plurality of segments based on the desired value of the level of stiffness, and
transmit a signal to the motor to cause the motor to adjust the effective bend length to the desired value.

2. The apparatus of claim 1, wherein the rotational axis aligns with a rotational axis of an ankle of a subject wearing the AFO frame and attached apparatus and wherein the plurality of segments of the elastic beam are parallel to a plantar-dorsiflexion (PD) plane such that the level of stiffness of the apparatus about the rotational axis is a level of stiffness in the PD plane about the rotational axis.

3. The apparatus of claim 1, further comprising a plurality of fulcrums, wherein each fulcrum defines a first opening to slidably receive a respective segment of the elastic beam, wherein the effective bend length of each segment is defined between an outer radial tip of each segment and the fulcrum and wherein the motor is configured to adjust the effective bend length of each segment based on adjustment of the position of the fulcrum along each of the plurality of segments.

4. The apparatus of claim 3, wherein the second frame portion comprises a plurality of guides that are oriented in the radial direction and wherein each fulcrum further defines a second opening to slidably receive a respective guide of the plurality of guides such that each fulcrum is configured to slide along each guide in the radial direction and is rotatably fixed to the second frame portion about the rotational axis.

5. The apparatus of claim 4, wherein the motor is operatively connected to each fulcrum such that the motor is configured to adjust a position of each fulcrum along each respective guide which causes adjustment of each fulcrum along each of the plurality of segments to adjust the effective bend length of each segment.

6. The apparatus of claim 4, further comprising a cam gear defining a plurality of arcuate slots, wherein each arcuate slot is configured to receive a portion of each fulcrum; wherein the motor is configured to rotate the cam gear such that the portion of each fulcrum slides within the respective arcuate slot and each fulcrum slides along each guide in the radial direction to adjust the effective bend length of each of the plurality of segments.

7. The apparatus of claim 6, further comprising a rotational disk operatively connected to the first frame portion, said rotational disk defining a plurality of radial slots, wherein each of the plurality of segments of the elastic beam comprises the outer radial tip extending in a direction orthogonal to the radial direction and wherein the outer radial tip of each of the plurality of segments is received in a respective radial slot of the plurality of slots such that upon rotation of the first frame portion relative to the second frame portion in a first direction the elastic beam is configured to impart a resistive moment in a second direction that is opposite to the first direction, said resistive moment based on the desired value of the level of stiffness.

8. The apparatus of claim 1, further comprising a rotational disk operatively connected to the first frame portion, said rotational disk defining a plurality of radial slots, wherein each of the plurality of segments of the elastic beam comprises an outer radial tip extending in a direction orthogonal to the radial direction and wherein the outer radial tip of each of the plurality of segments is received in a respective radial slot of the plurality of slots such that upon rotation of the first frame portion relative to the second frame portion in a first direction the elastic beam is configured to impart a resistive moment in a second direction that is opposite to the first direction, said resistive moment based on the desired value of the level of stiffness.

9. The apparatus of claim 1, further comprising:
a sensor configured to measure a value of a parameter of motion of a subject wearing the apparatus and AFO frame;
wherein the controller is communicatively coupled with the sensor such that the controller is configured to receive the measured value of the parameter and the controller is configured to determine the first data indicating the desired level of stiffness based on the measured value of the parameter.

10. A system comprising:
the apparatus of claim 1; and
the AFO frame comprising the shank portion and the foot portion.

11. A method comprising:
attaching a first frame portion to a shank portion of an ankle-foot orthosis (AFO) frame;
attaching a second frame portion to a foot portion of the AFO frame, wherein the second frame portion is configured to rotate relative to the first frame portion about a rotational axis when attached to the AFO frame;
providing an elastic beam comprising a plurality of segments oriented with a first length in a radial direction that is orthogonal to the rotational axis;
operatively connecting a motor to the plurality of segments, such that the motor is configured to adjust an effective bend length of each of the plurality of segments about the rotational axis, wherein the effective bend length is less than or equal to the first length;
receiving, at a controller, first data indicating a desired value of a level of stiffness about the rotational axis,
determining, with the controller, a desired value of the effective bend length of each of the plurality of segments based on the desired value of the level of stiffness;
transmitting, from the controller, a signal the motor a signal to cause the motor to adjust the effective bend length to the desired value;
adjusting, with the motor, the effective bend length of each of the plurality of segments to the desired value.

12. The method of claim 11, further comprising:
providing a plurality of fulcrums, wherein each fulcrum defines a first opening;
slidably receiving a respective segment of the elastic beam within the first opening of each fulcrum, wherein the effective bend length is defined between an outer radial tip of each segment and the fulcrum;
wherein the adjusting step comprises adjusting, with the motor, the effective bend length based on adjusting the position of the fulcrum along each of the plurality of segments.

13. The method of claim 12, wherein the second frame portion comprises a plurality of guides that are oriented in the radial direction and wherein the method further comprises:
slidably receiving a guide of the plurality of guides within a second opening of each fulcrum such that each fulcrum is slidable along each guide in the radial direction and each fulcrum is rotatably fixed to the second frame portion about the rotational axis.

14. The method of claim 13, wherein the operatively connecting step comprises operatively connecting the motor to each fulcrum and wherein the adjusting step comprises adjusting, with the motor, a position of each fulcrum along each respective guide which causes adjustment of each fulcrum along each of the plurality of segments and adjustment of the effective bend length of each segment.

15. The method of claim 13, further comprising:
providing a cam gear defining a plurality of arcuate slots;
slidably receiving a portion of each fulcrum in each arcuate slot;
wherein the adjusting step comprises rotating, with the motor, the cam gear such that the portion of each fulcrum slides within each arcuate slot and each fulcrum slides along each guide in the radial direction to adjust the effective bend length of each of the plurality of segments.

16. The method of claim 15, further comprising:
operatively connecting a rotational disk to the first frame portion, said rotational disk defining a plurality of radial slots;
receiving the outer radial tip of each segment of the plurality of segments of the elastic beam in a respective radial slot of the plurality of radial slots, wherein the outer radial tip of each segment extends in a direction orthogonal to the radial direction; and
imparting a resistive moment by the elastic beam in a second rotational direction upon rotation of the first frame portion in a first rotational direction that is opposite to the second rotational direction, wherein said resistive moment is based on the desired value of the level of stiffness.

17. The method of claim 11, further comprising:
operatively connecting a rotational disk to the first frame portion, said rotational disk defining a plurality of radial slots;
receiving a tip of each segment of the plurality of segments of the elastic beam in a respective radial slot of the plurality of radial slots, wherein the tip of each segment extends into each respective radial slot in a direction orthogonal to the radial direction; and
imparting a resistive moment by the elastic beam in a second rotational direction upon rotation of the first frame portion in a first rotational direction that is opposite to the second rotational direction, wherein said resistive moment is based on the desired value of the level of stiffness.

18. The method of claim 11, wherein the rotational axis aligns with a rotational axis of an ankle of a subject wearing the AFO frame and the first and second frame portions and wherein the plurality of segments of the elastic beam are in a plane that is parallel to a plantar-dorsiflexion (PD) plane such that the level of stiffness about the rotational axis is a level of stiffness in the PD plane about the rotational axis.

19. The method of claim 11, further comprising:
measuring, with a sensor, a value of a parameter of motion of a subject wearing the AFO frame with the attached first and second frame portions;
receiving, at the controller, the measured value of the parameter; and
determining, with the controller, the first data indicating the desired level of stiffness based on the measured value of the parameter.

20. A method for optimizing a value of a parameter of the plurality of segments of the elastic beam in the apparatus of claim 1, said method comprising:
determining a desired range of stiffness for the apparatus in order to treat one or more subjects having one or more medical diagnoses;
measuring a level of stiffness of one or more segments having one or more respective values of the parameter; and
selecting the plurality of segments for the apparatus and a respective value of the parameter for each of the plurality of segments of the apparatus, based on the measuring step, so that a range of the level of stiffness for the selected plurality of segments in the apparatus encompasses the desired range of stiffness.
